(19) United States
(12) Patent Application Publication
(10) Pub. No.: US 2024/0206740 A1
(43) Pub. Date: Jun. 27, 2024

(54) APPARATUS AND METHODS FOR EXTENDED INTRAORAL BODY TEMPERATURE MONITORING

(71) Applicant: Align Technology, Inc., San Jose, CA (US)

(72) Inventors: Byong-Ho PARK, San Jose, CA (US); Zijie ZHU, San Jose, CA (US); Sangshik PARK, San Jose, CA (US); Wesly WONG, Cupertino, CA (US); Fred TING, San Jose, CA (US); Hunho JO, San Jose, CA (US); Sheyda NAZARIAN, San Jose, CA (US); Spencer TODD, Folly Beach, SC (US); Eric E. KUO, San Jose, CA (US); Yaser SHANJANI, Milpitas, CA (US); Jun SATO, San Jose, CA (US); Srinivas KAZA, Mountain View, CA (US)

(21) Appl. No.: 18/396,679

(22) Filed: Dec. 26, 2023

Related U.S. Application Data
(60) Provisional application No. 63/435,221, filed on Dec. 23, 2022.

(51) Int. Cl. A61B 5/01; A61B 5/00; A61B 5/11; G16H 20/60

(52) U.S. Cl. CPC A61B 5/01; A61B 5/1118; A61B 5/4343; A61B 5/4812; A61B 5/4833; A61B 5/682; A61B 5/746; G16H 20/60; A61B 2562/0219; A61B 2562/0271

(57) ABSTRACT

Oral sensing apparatuses and methods for recording and tracking physiological parameters (e.g., intraoral temperature) over extended periods of time. These apparatuses can be part of an oral appliance, such as (but not limited to) an aligner, retainer, night guard, palatal expander, attachment, etc., or it can be made as a standalone device that can be directly attached to one or more teeth or an oral appliance. Also described herein are methods of extended monitoring of a subject intraoral temperature for a variety of different clinical and non-clinical applications.

### CLAIM OF PRIORITY

This patent application claims priority to U.S. Provisional Patent Application No. 63/435,221, titled “APPARATUS AND METHODS FOR EXTENDED INTRAORAL BODY TEMPERATURE MONITORING,” filed on Dec. 23, 2022, and herein incorporated by reference in its entirety.

### BACKGROUND

Body temperature, and in particular core body temperature, may be used as an indicator for a variety of conditions. For example, body temperature may indicate infection, fertility, and overall health. Temperature measurement may be used to detect disease or infection before it progresses. Periodic oral temperature sensing, e.g., using an oral thermometer inserted into the subject's mouth, has long been used as a diagnostic indicator for infection in clinical and home settings, but is generally understood to be of limited accuracy and may vary based on a number of different parameters. Detecting core body temperature directly is an invasive process and as such, it is difficult to monitor core body temperature for an extended period. As an alternative, temperature at a surface of the body (e.g., the external skin, the oral cavity) may be measured to indicate a peripheral temperature. In many instances, this peripheral temperature may be used to derive an approximation of core body temperature. Such peripheral temperature measurements are far less invasive, and may be preferred in many cases. In general, peripheral temperature measured from within the oral cavity corresponds more closely to, and varies less from, core body temperature than peripheral temperature measured along the external skin. However, although oral temperature sensing using devices such as oral thermometers is less invasive than core body temperature sensing, it may still be disruptive and infeasible for extended monitoring and may as a result be performed only infrequently.

It would be particularly useful to provide devices and techniques for extended measurement of body temperature, and in particular, devices that are comfortable, easy to use, and minimally invasive.

### SUMMARY OF THE DISCLOSURE

Described herein are intraoral apparatuses that can record and track intraoral body temperature over extended periods of time. The apparatus can be constructed as a part of an oral appliance, such as (but not limited to) an aligner, retainer, night guard, expander (e.g., palatal expander and/or lower-jaw expander), attachment, etc., or it can be made as a standalone device that can be directly attached to a tooth or a dental apparatus. Extended intraoral body temperature can be used as a biomarker for a variety of clinical applications. As used herein, extended intraoral temperature measurements may refer to measurements that are performed over an extended period that may last for one or more hours or one or more days. These measurements may be performed without removing the apparatus from the oral cavity. Measurements may be continuous, or may be discrete at regular or variable times, such as at between about every 0.1 second and every 10 minutes or longer (e.g., between about 10 Hertz and 1.67 milliHertz).

Long term monitoring may be used as a biomarker for one or more conditions. In some examples described herein, long-term monitoring of intraoral temperature may be used to detect and/or aid in treating a disease (e.g., for disease diagnosis) or disorder, such as sleep apnea, infection (e.g., influenza, COVID, etc.). Extended intraoral temperature monitoring may also or alternatively be used for sleep monitoring, metabolism monitoring or analysis, performance tracking, dietary activity monitoring, etc. Long term monitoring may include detection of one or more health indicators, including detection of elevated temperature (including fever).

In some of the examples described herein, thermal sensing may be performed with an oral thermal sensing device that is adapted so that it may be comfortably worn by the subject. In some cases these apparatuses may be configured so that they may be easily applied and/or removed by the subject. Thus, the apparatuses described herein may generally be adapted to be significantly easier to use than existing temperature sensing apparatuses, including oral appliances having temperature sensing. For example, described herein are sensing apparatuses (e.g., devices and systems) that may be configured as thermal sensing apparatuses that may be adapted for ease of insertion by the subject (e.g., patient) or caregiver, without requiring a dental professional to install and/or calibrate the apparatus. In particular, these apparatuses may include an elastic band that is configured so that it may be easily applied by the subject wearing the sensing device.

For example, described herein are sensor units configured as oral thermal sensing devices that include: a strap configured to attach to a tooth, wherein the strap includes an elastic region that is elastically adjustable; and a sensor unit coupled to the strap, the sensor unit comprising a thermal sensor, a power source, a communication circuit and a processor, wherein the processor is configured to receive temperature readings from the thermal sensor, and to store, transmit or store and transmit the temperature readings. In any of the methods and apparatuses described herein, the processor may be a remote processor or a local processor.

In general, an elastically adjustable strap may include one or more elastic regions that may stretch and contract so that they may adjust to fit a variety of different tooth sizes. The strap may adjust by virtue of the elastic regions. In some examples the strap may include one or more inelastic or less elastic regions that may be configured to provide strength while allowing some region of the strap (e.g., regions configured to extend between adjacent teeth) to be sufficiently thin so as to readily fit interproximally between the teeth. In some examples the strap may be a loop of material (e.g., forming a band that may encircle one or more teeth when worn). In some examples the strap may be convertible between an open configuration, in which the strap includes ends that are open until closed together to form a loop or band. In some examples the strap is always a closed loop that may be elastically expanded and/or contracted over the one or more teeth. In some examples the strap is configured to be cinched tighter or loosened by adjusting the connection between ends of the strap.

For example, the strap may have one or more interdental (e.g., interproximal) regions having a thickness of 0.2 mm or less. The interdental regions may be configured to fit between adjacent teeth. In some examples the strap includes one or more interdental regions configured to fit between a user's teeth and one or more elastic regions coupled to the interdental regions. The interdental regions may be less elastic than the one or more elastic regions. In some examples the interdental regions may be harder or stronger. These regions may also be thinner to facilitate placement between teeth.

Also described herein are oral sensing apparatuses that are configured so that the sensor unit is removably and/or replaceably engaged with an intraoral device including subject-removable intraoral devices, such as (but not limited to) an aligner, retainer, palatal expander, dental arch expander, etc. In particular, described herein are oral thermal sensing apparatuses that are configured so that the sensor unit may be removably attached to the intraoral device securely to prevent the separation of the sensor unit from the intraoral device, therefore minimizing choking risk, while preventing interference between the sensor unit and the operation of the intraoral device, which may itself be configured to controllably apply force to the subject's teeth and/or palate.

For example, described herein are systems for oral thermal sensing that include: a sensor unit comprising a housing having a flange extending at least partially around a base of the housing, wherein the housing at least partially encloses a thermal sensor and a processor that is configured to receive temperature readings from the thermal sensor, and to store, transmit, or store and transmit the temperature readings; and an intraoral device comprising a body forming a tooth-receiving cavity that is configured to fit over a subject's teeth, and an opening through a wall of the tooth receiving cavity, wherein the opening is configured to receive the housing of the sensor unit, so that the flange engages with an inner surface of the tooth-receiving cavity to secure the sensor unit in position when the intraoral device is worn on the teeth.

The opening may be configured to hold the base of the housing flush with the inner surface of the intraoral device. In some examples the opening is configured to hold the base of the housing in parallel with the inner surface of the intraoral device when the intraoral device is worn. For example, the base may include an inner lip or recess that receives and/or mates with the flange. In some cases this region may be configured to releasably engage with the flange. For example the opening may include a friction fitting (e.g., snap fit or clip-on fit) that may help secure the sensor unit to the opening. When assembled, e.g., by placing the sensor unit into the opening from within the tooth-receiving cavity, the flange may prevent the sensor unit from passing completely through the opening and the base on the back of the sensor unit may be held against the tooth. In some examples the contact with the outer surface of the tooth may help secure the sensor unit into the opening of the intraoral device.

The opening may extend completely through the intraoral device at any appropriate region of the intraoral device. In some examples the opening extends through a buccal side of the intraoral device when the intraoral device is worn. In some examples, the opening extends through a lingual side of the intraoral device when the intraoral device is worn.

The opening may comprise a recessed region that is recessed into the inner surface of the tooth-receiving cavity and configured to receive the flange of the intraoral device. The recessed region may be configured to engage with the flange. In some cases the recessed region is configured to seal with the flange and/or body of the sensor unit. This may prevent or reduce capturing food particles and/or contamination of the device.

The removable and/or replaceable devices described herein may allow the same sensor unit to be used with a plurality of different intraoral devices, including multiple devices of a treatment plan (e.g., a sequence of intraoral devices that may be used to treat a subject's teeth). In some examples the removable and/or replaceable device may be configured so that multiple sensor units may be used with the same intraoral device.

In any of these examples, the sensor unit may be a temperature sensor unit; in some cases this temperature sensor unit may be configured to measure a temperature having a sensitivity of 0.1 degree Celsius or a greater sensitivity (e.g., the temperature sensor may detect differences of at least 0.1 degree Celsius). Although the sensor units described herein are primarily described as temperature-sensing sensor units, any appropriate sensor unit may be used for monitoring a subject, including additional and alternative sensing modalities (e.g., pH, impedance, etc.). For example, any of these sensor units may further include one or more accelerometers configured to detect movement; these sensor units may therefore receive movement data to use, store, and/or transmit. Any of these sensor units may be configured to continuously record the temperature readings.

The intraoral device may comprise one or more of: an aligner, a retainer, and/or an expander (e.g., palatal expander, mandibular expander, etc.).

The sensor units (including oral thermal sensing devices) described herein may be configured to operate over an extended period so that thermal information from the oral cavity may be received continuously, at regular intervals, and/or at varying intervals. In some examples the rate that thermal information (e.g., temperature readings) are collected may vary depending upon one or more control variables, which may be preset or may be determined from prior thermal information and/or from one or more other sensors (e.g., motion sensors/accelerometers, etc.). For example, the oral sensing devices described herein may be configured to collect data more or less frequently when the subject is asleep, and/or when the subject is moving less. In some examples the sensor unit (e.g., oral thermal sensing devices) may be configured to collect thermal information at a rate that is dependent upon the time of day, based on a predetermined or adjustable schedule. In some examples the sensor unit (e.g., oral thermal sensing devices) descried herein may be configured to collect thermal information at a frequency that is dependent upon the power available to the oral thermal sensing device (e.g., reducing the rate of collection of thermal information when the battery of the oral thermal sensing device is low). In some examples, the sensor unit may adjust the rate of collection of thermal information in response to a request (e.g., command) received in a communications (e.g., wireless communications) circuit. Thus, in general, the sensor unit (e.g., oral thermal sensing devices) described herein may adjust the collection rate in response to an input.

Thus, any of the sensor unit (e.g., oral thermal sensing devices) described herein may include one or more processors that include control logic controlling the rate of collection and/or storage of thermal information.

In general, the strap may be configured to fit around one or more teeth. In some examples the strap may be configured to fit over just one tooth. The one or more teeth may be a premolar, molar, or canine tooth, and may be an upper tooth or a lower tooth. The strap may be configured to secure the sensor unit so that it may be comfortably worn without irritating the subject. For example, the oral thermal sensing device may be configured so that the strap holds the sensor unit against the cheek and/or gums in a region of the mouth that is relatively insensitive to irritation.

The oral thermal sensing devices described herein may be configured to hold to secure the sensor unit so that temperature is being sensed from one or more of; the gingiva, the inside check, saliva, and/or the air or fluid surrounding the teeth and gingiva. The oral thermal sensing device may include one or more thermal sensors, e.g., as part of the sensor unit. In some examples the sensor unit may be configured to sense temperature of the teeth (e.g., dentin). Teeth generally have poor heat transfer properties (e.g., their overall thermal conductivity may be about 0.6 W/m*K). Thus, the temperature of the teeth may be relatively insensitive to changes due to airflow (breathing) and/or consumption of food or drink in the oral cavity. Alternatively or additionally, the sensor unit may be configured to measure the temperature of gingiva. Alternatively or additionally, in some examples the sensor unit may be configured to measure the temperature of the gingiva. Alternatively or additionally, the sensor unit may be configured to measure the temperature of the open space within the oral cavity (e.g., by measuring along the lingual side of the teeth). In some examples the sensor unit may include multiple different thermal sensors arranged on different regions of the sensor unit to measure the temperature along one or more of check, gingiva, saliva, etc.

Any of the oral sensing devices, including oral thermal sensing units, described herein may be configured to engage with one or more attachments on the teeth. For example, the strap may comprise one or more engagements each configured to couple to an attachment bonded to one or more teeth. The one or more engagements may each comprise a snap-on attachment.

In general, the sensor unit (e.g., oral thermal sensing devices) described herein may be configured so that the sensor unit measures temperature with a sensitivity of 0.1 degree Celsius or greater (e.g., 0.009° C. or more, 0.008° C. or more, 0.007° C. or more, 0.006° C. or more, 0.005° C. or more, 0.002° ° C. or more, 0.001° C. or more, etc.). In some examples the sensitivity may be increased or decreased based on an input. For example, the sensitivity may be adjusted automatically or manually, e.g., in response to the frequency of measurements (e.g., at higher frequency measurements, a higher sensitivity may be used).

Any appropriate power source may be used for the sensor unit described herein. For example, the power source may comprise one or more of: a battery, a capacitor, and an energy harvesting circuit.

The sensor unit described herein may include or may be in communication with one or more additional sensors. For example, the sensor unit may also include one or more accelerometers configured to detect movement data and to store, transmit or store and transmit the movement data.

In general the sensor unit described herein may record absolute temperature or they may detect and/or record changes in temperature over time. For example, continuous recording may be taken by detecting changes (above a sensitivity threshold, e.g. 0.1° C., 0.09° C., 0.08° C., 0.07° C., 0.06° C., 0.05° C., 0.04° C., 0.03° C., 0.02° ° C., 0.01° C., 0.009° C., 0.008° C., 0.007° C., 0.006° C., 0.005° C., 0.004° C., 0.003° C., 0.002° C., 0.001° C., etc.). The sensitivity threshold may be determined or adjusted by the sensitivity of the sensor unit (e.g., of the thermal sensor).

As mentioned, in some examples the sensor unit of the oral thermal sensing device may be configured to continuously record the temperature readings. Under a continuous temperature sensing regimen, the apparatus (e.g., the oral thermal sensing device) may continuously monitor for changes in temperature that exceed the sensitivity threshold, and may record the change in temperature at a particular time. Tracking change in temperature over time may reduce the memory and/or power requirements.

In some examples an oral thermal sensing device may include: a strap configured to fit around a tooth, wherein the strap includes an elastic region that is elastically adjustable; and a sensor unit coupled to the strap, the sensor unit comprising a thermal sensor, a power source, a communication circuit and a processor, wherein the processor is configured to receive (e.g., continuously) temperature readings from the thermal sensor and to store, transmit or store and transmit the temperature readings. As just mentioned above, the temperature readings may be relative temperature readings (e.g., changes in temperature above a sensitivity threshold).

Also described are systems including any of the sensor unit (and in some cases the oral thermal sensing devices) described herein. For example, a system may include: an oral thermal sensing device comprising a strap configured to attach to a tooth, wherein the strap includes an clastic region that is elastically adjustable and a sensor unit coupled to the strap, the sensor unit comprising a thermal sensor, a power source, a communication circuit and a processor, wherein the processor is configured to receive temperature readings from the thermal sensor, and to store, transmit, or store and transmit the temperature readings; and a non-transitory computing device readable medium having instructions stored thereon that are executable by a processor to cause the processor to: receive intraoral thermal data from the oral thermal sensing device wherein the thermal data comprises sensor data collected by the oral thermal sensing device when worn in the subject's oral cavity; determine one or more conditions based on the intraoral thermal data; and output to a user display an indicator indicating the one or more conditions.

Any appropriate output to the user display may be used. The output may be textual (e.g., numeric, alphanumeric, icons), graphical, etc. In some examples the output to the user display may include a graphical display of the thermal data.

In general, the sensor unit described herein may be configured to conserve energy by storing data over the extended period that the device is worn in the oral cavity and may transmit at discrete times. In some examples, the oral thermal sensing device is configured to transmit the sensed temperature information to a remote processor for further analysis and/or output. The remote processor may include a handheld device (e.g., phone, tablet, etc.). The remote processor may include the non-transitory computing device readable medium having the instructions stored thereon to receive the intraoral thermal data.

In any of these systems, transmission between the oral sensing device and the remote processor may be via an intermediary, or bridging, device. The bridging device may coordinate communication between the oral sensing device and the one or more remote processors. In some cases the transmission of the temperature information (temperature readings) may be performed after the device is removed from the oral cavity. For example, the oral thermal sensing device may be removed and placed on the bridging device which may then transfer data from the oral thermal sensing device, encode it or otherwise process it, and transmit (or store and transmit) the oral thermal sensing data to the remote processor (e.g., phone and/or cloud-based processor, etc.). Thus, any of these systems may include a bridging device configured to couple the oral sensing device to the processor.

The bridging device may be configured to couple to the oral sensing device by near field communication (NFC) or by radio frequency identification (RFID). The bridging device may be further configured to communicate with the oral sensing device by Bluetooth, WiFi, or a physical cable connection.

Also described herein are methods of using any of the sensor units described herein. For example, a method may include: transmitting thermal data collected by an oral thermal sensing device when worn in a subject's oral cavity from the oral thermal sensing device to a bridging device; and transmitting the thermal data from the bridging device to a computing device. The method may further include establishing a communications link between the bridging device and the oral thermal sensing device, and/or establishing a communications link between the bridging device and the computing device. In any of these methods transmitting the thermal data from the oral thermal sensing device to the bridging device may include transmitting by near field communication (NFC) or by radio frequency identification (RFID). In some examples transmitting the thermal data from the bridging device to the computing device comprises transmitting by Bluetooth, WiFi or cable connection. In addition to transmitting sensed temperature information (and/or other sensor information) from the oral thermal sensing device, any of these systems and methods may be configured to transmit instructions from the computing device to the bridging device and from the bridging device to the oral thermal sensing device. For example, any of these methods may include instructing the oral thermal sensing device to turn on or off a sensor, and instructing the oral thermal sensing device to increase or decrease the frequency of sensor data acquisition.

The apparatuses described herein may include one or more features that make them more effective in processing, analyzing and or presenting temperature information. For example, also described herein is software (e.g., non-transitory computing device readable medium having instructions stored thereon) that may receive and process thermal data from any of the sensor units described herein or other sensor unit. In some examples the software (or firmware) may be configured as an application software for use on a smartphone or tablet, including in particular the subject's smartphone, on a desktop/laptop computer, etc.

For example, described herein are non-transitory computing device readable medium having instructions stored thereon that are executable by a processor to cause the processor to: receive intraoral thermal data from an oral thermal sensing device, wherein the oral thermal sensing device is worn in a subject's oral cavity, and wherein the thermal data comprises sensor data collected by the oral thermal sensing device; determine one or more conditions based on the intraoral thermal data; and output to a user display an indicator indicating the one or more conditions.

The instructions may be configured to receive the intraoral thermal data from the oral thermal sensing device. In some examples the intraoral thermal data is collected continuously (e.g., for more than 5 minutes, 10 minutes, 15 minutes, 20 minutes, 30 minutes, 45 minutes, 1 hour, 2 hours, 3 hours, 4 hours, 6 hours, 12 hours, 1 day, etc.).

In some examples the instructions are configured to receive the intraoral thermal data from the oral thermal sensing device, wherein the intraoral thermal data is collected continuously at frequency of 0.5 Hz or greater while the oral thermal sensing device is worn in the subject's oral cavity, and wherein the intraoral thermal data has a sensitivity of 0.1 degree Celsius or greater.

In some examples the one or more conditions comprises a sleep condition. For example, the instructions may be configured to identify one or more sleep/wake cycles based on the received intraoral thermal data and to determine a sleep abnormality condition based on the identified sleep wake cycles. The instructions may be configured to identify the sleep/wake cycle from the received intraoral thermal data by identifying a low-frequency cyclical decrease in the intraoral temperature. In some examples the low-frequency cyclical decrease has a frequency of less than 0.0033 Hz.

Also described herein are non-transitory computing device readable medium instructions adapted to determine, track and/or monitor a longevity fitness condition. The longevity fitness condition may be based on a correlation between a core body temperature derived from the intraoral thermal data and a time of day.

In some examples the non-transitory computing device readable medium instructions are configured to determine a consumption of a food or beverage condition. For example, the consumption of a food or beverage condition may be determined by identifying a pattern indicating eating and/or drinking from the intraoral thermal data. The apparatus may determine and/or record (e.g., log) when a subject is eating/drinking and output an indicator, such as displaying it visually (e.g., on the subject's smartphone). In some examples the system may analyze and make a determination as to whether the subject is conforming to or is deviating from a diet plan.

Any of the apparatuses (e.g., the non-transitory computing device readable medium instructions) may detect and/or track physical activity, such as exercise, based on a change in temperature characteristic of exercise. In some examples this can be presented on the same app/interface as the diet information.

In some examples the non-transitory computing device readable medium instructions are configured to detect a fertility condition, such as ovulation, pregnancy, etc. For example, the fertility condition may be detected by identifying a pattern for ovulation and/or pregnancy from the intraoral thermal data.

The non-transitory computing device readable medium instructions may be configured to detect an infection condition. For example, the instructions may be configured to determine that the subject is likely to have an infection condition based on the received intraoral thermal data. In some examples the system (e.g., the instruction of the non-transitory computing device-readable medium instructions) may be configured to distinguish between one or more different types of infection. For example, the system may be configured to distinguish between a first infection condition that has a first temperature profile and a second infection condition that has a second temperature profile. The instructions may be configured to determine that the subject has the first infection condition based on determining that the subject has a core body temperature conforming to the first temperature profile and not the second temperature profile. For example, the first infection condition may be COVID-19 and the second infection condition may be influenza.

In general, these methods and apparatuses, an in particular the non-transitory computing device readable medium instructions, may be configured to detect the one or more conditions from the pattern of the intraoral thermal data using a trained machine learning network.

As mentioned, any of the oral sensing devices described herein may be used with any the software described herein. For example, a system for continuously monitoring intraoral temperature, may include: an oral thermal sensing device; and a non-transitory computing device readable medium having instructions stored thereon that are executable by a processor of a computing device (e.g., a mobile telecommunications device, a laptop/desktop computer) to cause the computing device to: establish a communication link with the oral thermal sensing device; receive intraoral thermal data from the oral thermal sensing device (wherein the data is received while the oral thermal sensing device is worn); output, to a user display an indicator indicating the communication link; determining one or more conditions from a pattern of the intraoral thermal data; and output to the user display an indicator indicating the one or more conditions. In some examples the oral thermal sensing device may be configured to transmit the intraoral thermal data to the computing device continuously or periodically (in regular or non-regular intervals). In some embodiments, the oral thermal sensing device may be configured to transmit the intraoral thermal data while the oral thermal sensing device is being worn.

In some examples, the intraoral thermal data is transmitted periodically (e.g., rather than continuously while the device is in the mouth). For example, the thermal data may be transmitted to an intermediary device (which may be configured as a case), e.g., via NFC, and the bridging device can transmit to a phone via Bluetooth/Wi-Fi. In some examples the intraoral thermal data may be transmitted continuously, e.g., via Bluetooth/wireless communication, while the device is worn. Also described herein are customized sensor unit (e.g., oral thermal sensing devices) and method for customizing them. In particular, described herein are methods for customizing oral thermal sensing devices and/or an intraoral device including or coupled to a sensor unit so that they may both be worn comfortably for extended periods and so that they may reliably and predictably position the one or more sensor units. Proper positioning of the sensor unit (and therefore the thermal sensors) may be particularly helpful in achieving reliable and accurate temperature sensing, particularly when measuring core body temperature. Customization may be applied to the oral thermal sensing devices having an elastically adjustable band and/or to oral thermal sensing devices having shell bodies including one or more cavities for holding the subject's teeth (e.g., similar to shell aligners).

For example, described herein are methods that include: receiving a scan of a subject's oral cavity; determining, from the scan, a position on an oral appliance for a sensor unit, so that the sensor unit minimally contacts and/or minimally interferes with the subject's tongue and gingiva when the sensor unit is coupled to the oral appliance and the oral appliance is worn by the subject; and outputting instructions for forming the oral appliance configured to hold the sensor unit in the determined position.

Also described herein are method of determining that a subject has an elevated temperature. In some examples, these methods may determine if the subject is feverish. For example, a method may include: receiving temperature sensor data from a sensor unit worn on one or more teeth in a subject's oral cavity; confirming that the subject is wearing the sensor unit; confirming that the temperature reading does not correspond to an environmental influence; determining if a subject has an elevated temperature by comparing the temperature sensor data to an elevated temperature detection threshold based; and triggering an alert indicating that the subject is has an elevated temperature if the temperature sensor data is greater than the fever detection threshold. In some cases the elevated temperature threshold is a fever threshold, and the alert may be triggered if the subject is feverish.

In any of these methods, the sensor unit may be attached to the subject's tooth or teeth directly (e.g., using a strap or band, etc.) or may be part of or attached to (including removably attached to) an intraoral device, such as subject-removably intraoral device (e.g., aligner, expander, retainer, etc.). For example, receiving may comprise receiving temperature sensor data from the sensor unit worn a removable dental appliance that is worn on the subject's teeth. In some cases the sensor unit may be worn on one or more of: an aligner, a retainer, and/or a palatal expander.

Any of these methods may confirm that the subject is wearing the sensor unit, and in particular, may confirm that the subject is wearing the sensor unit in the oral cavity. For example, the method (or an apparatus configured to perform the method) may include confirming an impedance measurement corresponding to the sensor unit is within an expected range of values corresponding to the oral cavity. One or more other sensors may be used to confirm that the sensor unit is within the mouth, and/or in contact with one or more teeth, such as an optical sensor, electrical sensor, conductivity sensor, etc.

In general, these methods may confirm that the temperature reading taken by the sensor unit corresponds to a subject's body temperature and not to one or more environmental influences, such as what the subject is eating (e.g., cold/hot food), drinking (cold/hot beverages), breathing (open mouth breathing), ambient temperature (e.g., when removed from the mouth), etc. For example, the method may confirm that the temperature reading does not correspond to an environmental influence by confirming that the subject is not eating, drinking, or engaged in strenuous exercise (e.g., increased respiration which may artificially reduce the temperature within the mouth); this may be confirmed based on the stability of the temperature reading and/or one or more additional sensors. In general, any of these methods may determine or confirm either or both the stability of the temperature reading over time and/or an activity sensor (e.g., accelerometer, etc.).

The elevated temperature detection threshold may be based on the temperature baseline. The temperature baseline may be derived from longitudinal data taken from the subject over time, as described herein. The baseline may be based on temperature data taken during periods of actual and/or expected low activity, such as during sleeping periods (e.g., between midnight and 6 am), and/or based on activity sensors (e.g., accelerometers) within the sensor unit. In some examples the longitudinal data is taken over a month or more.

The elevated temperature detection threshold may comprise the baseline oral temperature plus an offset temperature. For example, the offset temperature may be about 0.4 degrees C., about 0.5 degrees C., about 0.6 degrees C., about 0.7 degrees C., about 0.8 degrees C., about 0.9 degrees C., about 1.0 C, about 1.1 degrees C., about 1.2 degrees C., about 1.3 degrees C., about 1.4 degrees C., about 1.5 degrees C., about 1.6 degrees C., about 1.7 degrees C., about 1.8 degrees C., etc. For example, the offset temperature may be about 0.5 degrees C. or more. Alternatively or additionally, the elevated temperature detection threshold may be based on one or more of: a time of day of the received temperature sensor data and/or on the subject's age.

Any of these methods may include forming the oral appliance using the instructions. For example, the oral appliances may be formed by a three-dimensional (3D) printing technique.

In some examples determining the position on the oral appliance for the sensor unit comprises using a trained neural network to determine the position. For example, the trained neural network may be trained on a dataset including a plurality of oral cavity scans and associated subject-reported comfort data.

Any of these methods may include receiving a model of the oral appliance. The oral appliance may be any appropriate oral appliance, such as but not limited to an aligner, a palatal expander, etc.

The methods described herein may include receiving a treatment plan comprising one or more oral appliances.

Outputting instructions may comprise outputting an instruction file (e.g., digital file) for forming the oral appliance configured to hold (or including) the sensor unit. In some examples, outputting instructions comprises outputting a digital model of the oral appliance.

Also described herein are systems configured to perform any of the methods described herein, including systems for customizing an oral thermal sensing device including a sensor unit. For example, described herein is a non-transitory computing device readable medium having instructions stored thereon that are executable by a processor to cause the processor to: receive a scan of a subject's oral cavity; determine, from the scan, a position on an oral appliance for a sensor unit, so that the sensor unit minimally contacts and/or minimally interferes with the subject's tongue and gingiva when the sensor unit is coupled to the oral appliance and the oral appliance is worn by the subject; and output instructions for forming the oral appliance configured to hold the sensor unit in the determined position.

The instructions may be further configured to cause the processor to form the oral appliance using the instructions. In some examples the instructions may cause the processor to form the oral appliance by three-dimensional (3D) printing. The instructions may cause the processor to determine the position on the oral appliance for the sensor unit using a trained neural network to determine the position. For example, the trained neural network may be trained on a dataset including a plurality of oral cavity scans and associated subject-reported comfort data. The instructions may cause the processor to receive a model of the oral appliance. As mentioned above, in some examples the oral appliance comprises an aligner, palatal expander, etc.

The methods and apparatuses described herein may be used with, and may incorporate or extend, the methods and apparatuses described in U.S. provisional patent application 63/579,502, titled “INTRAORAL APPARATUSES CONFIGURED TO MONITORING USE,” filed on Aug. 29, 2023, which is herein incorporated by reference in its entirety.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates two examples of oral sensing devices including a sensor unit that is coupled to an intraoral appliance, in this example an aligner.

FIGS. 2A-2B show examples of sensor units that may be used as part of an oral thermal sensing device.

FIG. 3 shows an example of an oral thermal sensing device including a sensor unit that is affixed to an oral appliance (e.g., aligner or palatal expander) configured to be removably worn by a subject.

FIG. 4 schematically illustrates one method of customizing an oral thermal sensing device as described herein.

FIG. 5A schematically illustrates an example of an oral thermal sensing device configured to couple to attachments on a subject's teeth.

FIG. 5B schematically illustrates the oral thermal sensing device of FIG. 5A coupled to a subject's teeth.

FIG. 5C illustrates an oral thermal sensing device such as that shown in FIG. 5A coupled to the subject's teeth.

FIG. 5D shows an example of an intraoral mount for a removable/replaceable self-locking sensor unit.

FIG. 6A schematically illustrates an example of an oral sensing device configured to couple to a subject's teeth.

FIG. 6B schematically illustrates the oral sensing device of FIG. 6A coupled to a subject's dentition.

FIG. 7 schematically illustrates an example of an oral sensing device including an elastically adjustable band.

FIG. 8A schematically illustrates an example of an oral sensing device including an elastically adjustable band.

FIG. 8B shows an example of an intraoral mount for a removable/replaceable self-locking sensor unit.

FIG. 9 schematically illustrates the oral sensing device of FIG. 7 attached to a subject's teeth.

FIG. 10A schematically illustrates an example of an oral sensing device configured to couple to a subject's teeth.

FIG. 10B schematically illustrates the oral sensing device of FIG. 10A coupled to a subject's dentition.

FIGS. 11A-11B schematically illustrates examples of oral sensing devices configured to couple to a subject's teeth by a cap or cuff.

FIG. 11C schematically illustrates an example of an oral sensing device similar to those shown in FIGS. 11A-11B, coupled to a subject's tooth.

FIG. 11D shows an example of an intraoral mount configured as a cap or cuff for a removable/replaceable self-locking sensor unit.

FIGS. 12A-12C are graphs showing temperature reading recorded from an oral thermal sensing device similar to that shown in FIG. 1. FIG. 12B shows an expanded view of a first (A) region of FIG. 12A. FIG. 12C shows an expanded view of a second (B) region of FIG. 12A.

FIG. 13 is an example of a graph showing temperature readings illustrating changes in body temperature reflecting various fertility conditions.

FIG. 14 is an example of a user interface.

FIGS. 15A-15B illustrate examples of sensor units that are configured to lock into an oral appliance (e.g., a removable oral appliance).

FIGS. 16A-16B illustrate one example of a sensor unit that is configured to removably lock into an oral appliance. FIG. 16A shows a system including the sensor unit and the oral appliance configured to removably receive the sensor unit in a self-locking configuration. FIG. 16B shows the system of claim 16A assembled.

FIGS. 17A-17B illustrate examples of oral appliances including one or more removable/replaceable self-locking sensor units. FIG. 17A show a system including a sensor unit coupled to an orthodontic retainer. FIG. 17B shows a system including a sensor unit coupled to a palatal expander apparatus.

FIGS. 18A-18B illustrate an example of a system including a removable and/or replaceable sensor unit and a dental aligner. FIG. 18A shows the system with the sensor unit separate from the dental aligner, and FIG. 18B shows the system with the sensor unit coupled to the dental aligner.

FIGS. 19A-19B schematically illustrate methods of determining oral temperature using any of the apparatuses described herein. FIG. 19A shows an example of a generic method of determining oral temperature using a sensor unit. FIG. 19B shows one example of a method of determining oral temperature.

FIGS. 20A-20B schematically illustrate methods of determining a baseline for oral temperature using any of the apparatuses described herein. FIG. 20A shows an example of a generic method of determining baseline oral temperature using a sensor unit. FIG. 20B shows one example of a method of determining baseline oral temperature.

FIGS. 21A-21C schematically illustrate methods of detecting an elevated temperature (e.g., in some cases, a fever) using the oral sensors described herein. FIG. 21A shows an example of a generic method of detecting an elevated temperature (including, but not limited to, a fever).

FIG. 21B shows an example of a first method of detecting an elevated temperature using any of the apparatuses described herein. FIG. 21C shows an example of a second method of detecting an elevated temperature as described herein.

FIG. 22 is graph showing raw data from a sensor worn within a subject's oral cavity in which the sensor unit is coupled to an intraoral appliance that is worn by the subject.

FIG. 23 is graph showing a comparison of baseline temperature and body temperature for a subject wearing an intraoral appliance including a sensor unit as described herein.

FIG. 24 is graph showing the duration of periods of low, normal and high temperature, as compared to baseline body temperature, using an intraoral appliance including a sensor unit that is worn by the subject.

FIG. 25 is a graph showing periods of high temperature (e.g., greater than a subject-specific baseline) over a period of three weeks.

FIG. 26 is a graph showing the duration of periods of time in which a subject wearing an oral appliance is either below or above a baseline temperature by a threshold value.

FIG. 27 shows an example of a sensor unit removably coupled to an intraoral mount that is worn on a subject's tooth.

FIGS. 28A-28B show side perspective and side views, respectively of an example of mating components that may be used to couple a sensor unit to an intraoral mount; in this example the mating components are configured to form a snap button connector.

FIGS. 29A-29B show side perspective and side views, respectively of an example of mating components that may be used to couple a sensor unit to an intraoral mount; in this example the mating components are configured to slide in relative to each other until a snap-in connection is engaged.

FIGS. 30A-30B show side perspective and side views, respectively of an example of mating components that may be used to couple a sensor unit to an intraoral mount; in this example the mating components are configured as a rotation lock.

FIG. 31A shows a partial section view through an example of mating components that may be used to couple a sensor unit to an intraoral mount.

FIG. 31B illustrates one example of a sliding slot connector, and FIG. 31C shows an example of a sliding connector with a snap connection, either of which may be used to couple a sensor unit to an intraoral mount.

### DETAILED DESCRIPTION

Described herein are apparatuses and methods for intraoral temperature sensing and measurement, as well as apparatuses and methods for using intraoral temperature sensing to detect, diagnose, and/or monitor one or more conditions.

In general, intraoral body temperature may be detected and used as a biomarker for various applications, e.g., sleep monitoring, COVID detection, life longevity, etc. Intraoral body temperature has been used to measure and detect fever due to illness such as cold and flu. However, extended measure of intraoral temperature has proven challenging, at least in part because it is cumbersome and uncomfortable to place a thermometer inside the mouth for a long period of time. The intraoral apparatuses (devices and systems) and methods described herein may monitor intraoral body temperature for extended period both accurately and comfortably. These apparatuses may generally include a temperature sensor, a memory, and a power source (e.g., a battery, capacitor, an energy harvesting circuit (TEG, piezoelectric)). In some examples, the apparatuses may include a mount subsystem, e.g., “mount,” for coupling within the subject's oral cavity.

The mount for securing the oral sensing device to the subject's teeth may be a dental appliance such as an aligner having one or more cavities for holding teeth. Examples of an aligner are described in detail in U.S. Pat. No. 5,975,893, and in published PCT application WO 98/58596, which is herein incorporated by reference for all purposes. Systems of dental appliances employing technology described in U.S. Pat. No. 5,975,893 are commercially available from Align Technology, Inc., Santa Clara, Calif., under the tradename, Invisalign System. Throughout the description herein, the use of the terms “orthodontic aligner”, “shell aligner”, or “dental aligner” may be synonymous with the use of the term aligner. Other mounts for securing the oral sensing device to the subject's teeth may be configured as palatal expanders, retainers, mouthguards, night guards, or other subject-insertable or subject-removable dental appliances.

In some embodiments, the mount may be an element that is configured to secure the oral sensing device directly to one or more teeth (e.g., without requiring a separate dental appliance). For example, the mount may include a strap, attachment, fastener, or any other suitable element that secures the oral sensing device at a desired location against one or more teeth. A few examples of such mounts are disclosed herein.

An oral sensing device may generally include one or more sensor units and a support, frame, or holder for securing the one or more sensor units within the oral cavity. The oral sensing device may include a strap or band for directly coupling the sensor unit to the tooth or teeth and/or may include an intraoral device (e.g., aligner, expander, retainer, etc.) to which the sensing device is coupled. For example, any of the oral sensing devices described herein may include a sensor unit that is either coupled to or configured to couple to the mount. A sensor unit may include one or more thermal sensors, a power source, a communication circuit, and one or more processors. The one or more processors may be configured to receive temperature readings from the one or more thermal sensors, and to store and/or transmit (e.g., store, transmit, or store and transmit) the temperature readings. In some examples the sensor unit may be configured to be removably coupled to the mount. Alternatively, in some examples the sensor unit may be configured to be permanently attached to the mount. In some examples a plurality of different mounts may be used with the same sensor or with different sensors. The combination of the sensor unit and the mount may be collectively referred to as an oral sensing device.

In any of the examples described herein the oral sensing device may be an oral thermal sensing device and may include one or more thermal sensor. Alternatively or additionally one or more non-thermal sensors may be included, including electrical sensors (e.g., impedance), optical sensors, vibration sensors, chemical sensors, pH sensors, etc.

As used herein, a processor may include hardware that runs the computer program code. Specifically, a processor may include a controller and may encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

Any of the sensor units described herein may include a housing (e.g., an outer housing) that may at least partially contain the components of the sensor unit, may protect the components, and/or may direct the input into the one or more thermal sensors. The housing may be formed of a biocompatible material. In some examples the housing is formed of the same material as the mount (e.g., an aligner, palatal expander, retainer, mouthguard, night guard, etc.).

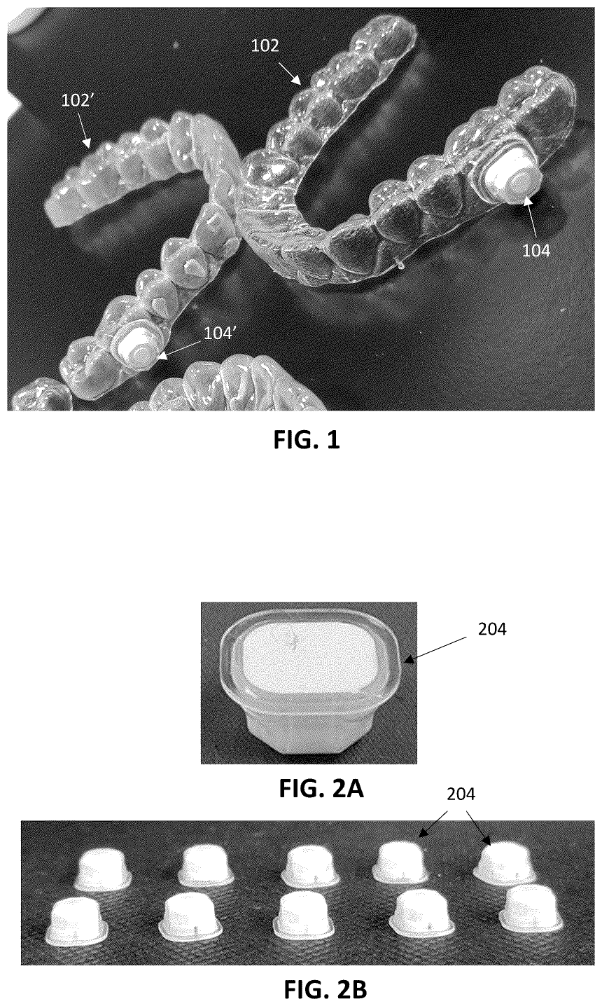

For example, FIG. 1 illustrates one example of a pair of oral sensing devices, configured as oral thermal sensing devices, each including a mount configured as an aligner 102, 102′ and a sensor unit 104, 104′ attached to the mount. In FIG. 1, the sensor unit is shown encapsulated in a polymeric housing material, and is mounted to an intraoral appliance, e.g., an aligner. As described herein, the position of the sensor unit on the mount may be selected to maximize both subject comfort and sensitivity for detecting the desired thermal signal. As will be described in greater detail below, the position of the sensor unit may be customized to the subject, based on either or both the intended purpose (e.g., sensing core temperature, temperature of saliva, gingiva, teeth, oral cavity, etc.), and/or based on the specific subject anatomy. In some embodiments, a subject may only wear one oral sensing device. In other embodiments, the subject may wear two or more oral sensing devices as described below. For example, as illustrated in FIG. 1, there may be two sensor units 104 and 104′, one attached to a top intraoral appliance and the other attached to a bottom intraoral appliance.

Alternatively or additionally, the oral sensing device may be configured to include a mount having more than one position and/or orientation for the sensor unit, so that the subject may themselves adjust the position and/or orientation of the sensor unit to optimize the comfort. For example, the mount may be an aligner, palatal expander, retainer, mouthguard, or night guard having a plurality of different predetermined coupling regions for coupling to a sensor unit. These predetermined coupling regions may be configured to hold the sensor unit in an orientation so that the temperature sensor of the sensor unit is exposed to the region of the oral cavity (e.g., teeth, gingiva, etc.) to be sensed.

In some examples the sensor unit may be configured to be releasably or permanently coupled to a permanent implant, such as a tooth implant, denture, etc. In some examples, the sensor unit may be made integral to such permanent implant. In some examples, the sensor unit may be configured to be releasably or permanently coupled to a tooth.

In some examples the temperature sensor unit (which may equivalently be referred to herein as a sensor unit) can be encapsulated with a biocompatible material, as shown in FIGS. 2A-2B, and the encapsulated device can be mounted to an intraoral appliance as shown in FIG. 1. As mentioned, FIG. 1 shows a pair of aligners 102, 102′ each with an encapsulated sensor unit 104, 104′. In some embodiments, the sensor units may be configured to be removably attached to an oral appliance. In some embodiments, the sensor units may be formed to be integral with an oral appliance. For example, the sensor units may be overmolded on an oral appliance (e.g., an aligner, a retainer, a palatal expander). Multiple sensor units can be mounted to a mount, including to an aligner, to measure temperature in multiple locations. Alternatively or additionally, the same sensor unit may include multiple thermal sensor inputs at different locations, such as on the back face, top, bottom, sides and/or front. For example, sensing the temperature of a tooth or teeth may be made from the back of the sensor unit, sensing from the gingiva may be performed from the sides or bottom and/or sensing from the check may be sensed from the front of the sensor unit. In some examples the same senor unit may have multiple sensor inputs at different locations and/or orientations, allowing for different temperature measurements that may be used to detect or diagnose physiological symptoms or behaviors. Alternatively or additionally, multiple different sensor units may be used. For example, one sensor unit can be placed in a sublingual area (under the tongue) and another sensor unit can be placed between a tooth and a check skin. The difference in the temperatures measured by these units can indicate if a user is breathing through the nose rather than the mouth during sleep or a physical activity. In some examples, the sensor unit can be simplified for easy placement and less interference with daily life. As another example, a one sensor unit may be positioned on a buccal side of a tooth and another sensor unit may be positioned on a lingual side of a tooth (e.g., the same tooth or a different tooth). As another example, a first sensor unit may be positioned on a molar on a left side of the subject and a second sensor unit may be positioned on a molar on a right side of the subject.

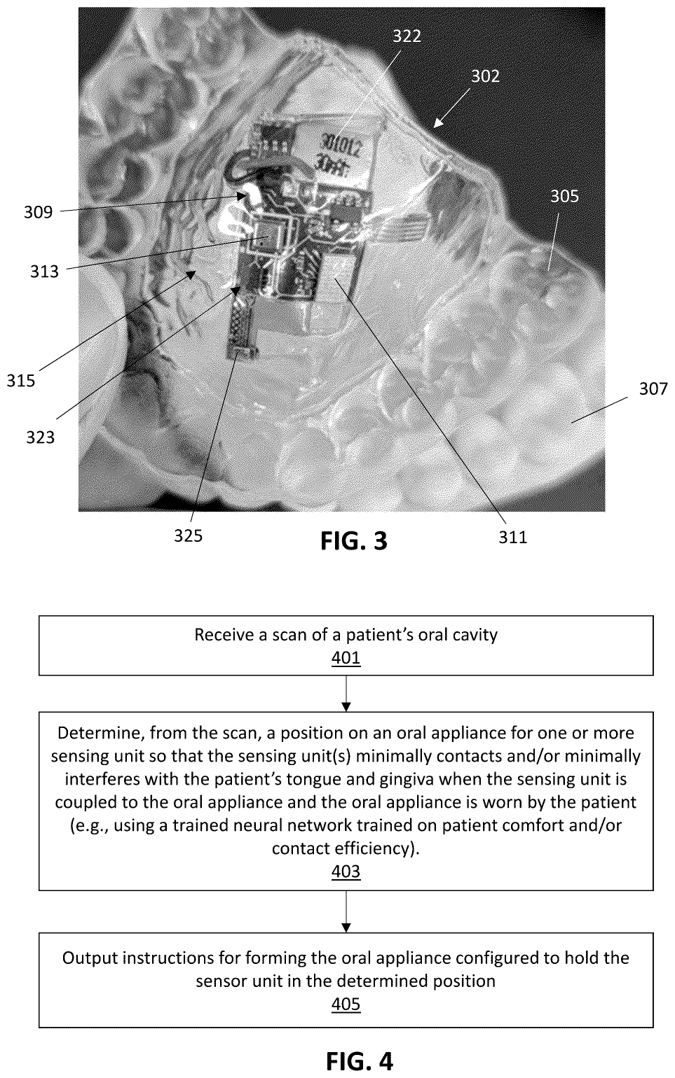

In some examples, such as that shown in FIG. 3, the oral sensing device 302 may include a mount formed as an intraoral appliance configured as an aligner 305 (e.g., a thermoformed aligner, a 3D printed aligner), or alternatively as a palatal expander, having a palatal area 315, one or more cavities for holding teeth (e.g., that may fit over the teeth or a model of the teeth 307) and the sensor unit is shown embedded or built into the palatal region 315. The oral sensing device 302 has a sensor unit with various functions and components that are visible in this example, including a temperature sensor 309, wireless communication circuitry 311, a pressure sensor 313, a battery 322, a motion sensor 323, and a force sensor 325, all shown embedded into the aligner 302.

As mentioned above, in general, these apparatuses may be customized to position the sensor unit (or sensor units) relative to the mount (e.g., aligner, palatal expander, retainer, mouthguard, night guard, etc.) for comfort and/or for efficacy. FIG. 4 illustrates one example of a method of customizing as described herein. As shown in FIG. 4, customization of the position and/or orientation of the sensor unit(s) may include using a scan or model, typically a digital model and/or digital scan of the subject's oral cavity, which may include the teeth and or/gingiva. In some examples, the scan or model may additionally include the tongue, check(s), and/or palate. As an initial step, the method or a system configured to perform the method may first receive the model or images (e.g., scan) of the subject's oral cavity 401. The system or method may then determine where on the mount to position the sensor unit(s) based on the subject anatomy from the scan and/or based on which portion of the oral cavity that is to be monitored for temperature. The method may also include inserting the identity of the type of mount (e.g., the type of oral appliance).

For example, the method (or an apparatus configured to perform the method) may then determine, from the scan or other model of the subject's oral cavity, a position on the oral appliance for the sensor unit, so that the sensor unit minimally contacts and/or minimally interferes with the subject's tongue and gingiva when the oral sensing device is coupled to the oral appliance and the oral appliance is worn by the subject 403.

In any of these examples a neural network (e.g., a machine learning agent) may be trained to determine what positions and/or orientations on the dental (oral) appliance are best suitable based on a subject scan data and the target region of the oral cavity to be monitored (e.g., gingiva, teeth, etc.). For example, a training dataset may include a scan and oral thermal sensing devices with one or more thermal sensor units on them that are annotated or otherwise indicate comfort of the subject and/or sensitivity or efficacy of the thermal sensor with the sensor unit in the particular position given the subject anatomy. The training dataset may include a type or category of the oral appliance, e.g., mount, to which the sensor unit it coupled.

Based on the output of the analysis (e.g., the output of the trained network), a proposed position and/or orientation of the sensor unit may be identified, and output guidance may be provided, e.g., outputting instructions forming the oral appliance configured to hold the sensor unit in the determined position 405. In some embodiments, the output instructions may be enumerated instructions that include specific parameters for forming the oral appliance. In some embodiments, the output instructions may be a guide or model (e.g., a virtual/digital model) of the oral appliance. In some examples the instructions may include instructions for forming or coupling the sensor unit to the oral appliance. For example, the instructions may include a generated model.

### Example Devices

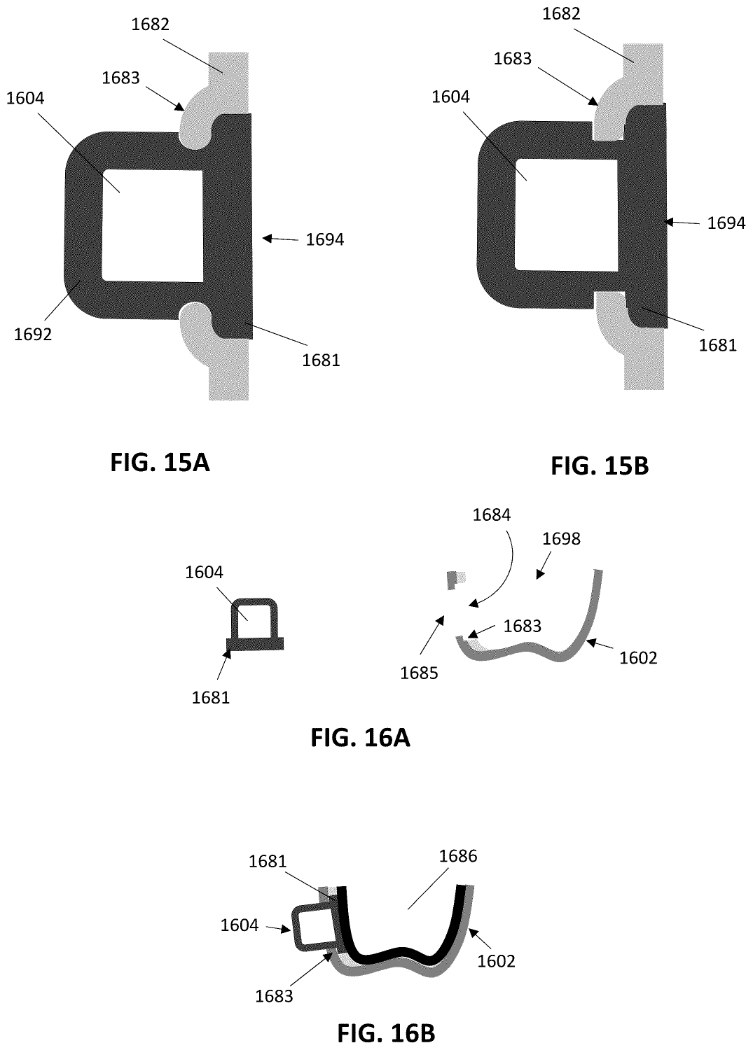

As mentioned above, and described in greater detail below, including in reference to FIGS. 15A-15B, 16A-16B, 17A-17B and 18A-18B, the sensor unit may be releasably or replaceably connected to an intraoral device. In addition, any of the oral sensing devices described herein may be configured as stand-alone devices that can be attached to and removed from a structure inside the mouth (tooth, implant, gum, palate, etc.). In some examples the oral sensing device can be clipped onto or wrapped around a tooth. In particular, these apparatuses may be configured so that the sensor unit and/or the oral appliance have one or more mating features so that the sensor unit can be easily coupled with (e.g., snapped onto) the oral appliance for secure placement.

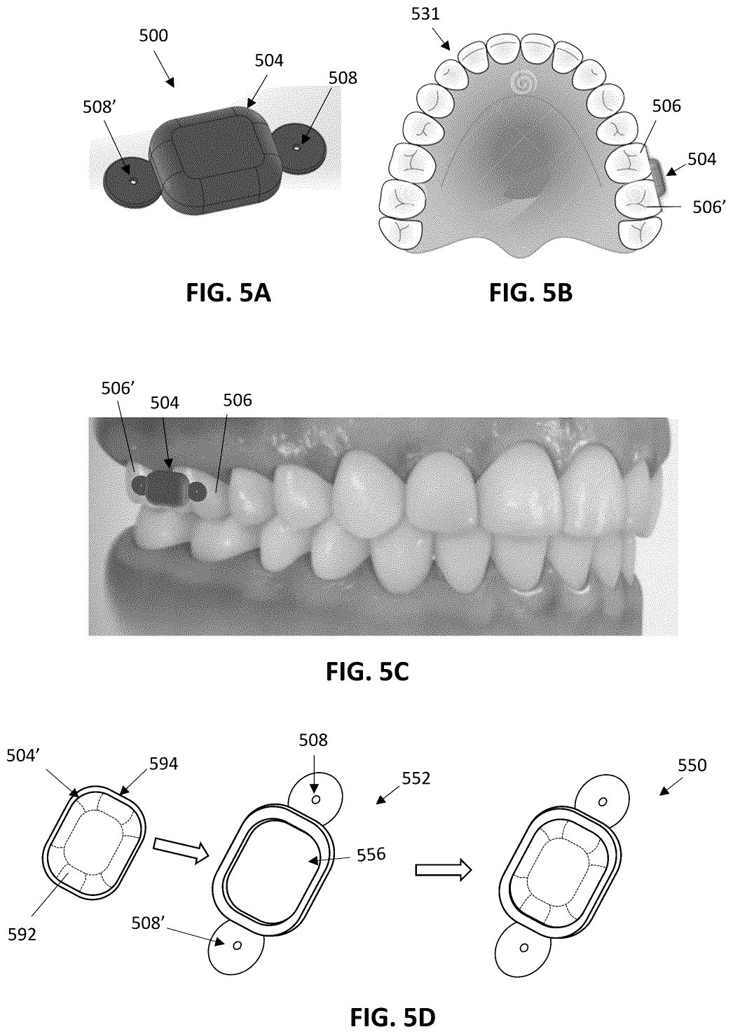

In some examples the oral sensing device includes a strap as part of a mount. The device can be a standalone device that can be clipped, snapped, placed around, or otherwise secured onto a structure (e.g., a tooth, a set of teeth, an implant, gingiva, tissue) inside the mouth. For example, an oral sensing device having a strap can be attached to a tooth (or multiple teeth) or placed and formed around a palatal area as shown in FIGS. 5A-5C. In this example, the oral sensing device 500 can be removed when it needs to be washed, when teeth need to be brushed, or for any other purpose, but may otherwise remain securely attached to the teeth (or a tooth). As shown in FIG. 5A, the device 500 includes an enclosed sensor unit 504 and a pair of anchor attachments 508, 508′ on either side of the sensor unit. In one example the tooth or teeth to which the device is to be attached can be configured to receive the device. For example, the tooth 506 and/or the tooth 506′ can include one or more anchors (e.g., attachments) on the surface of the tooth (or teeth) so that the device may be easy to attach and remove the device with a mating feature, e.g., anchor attachments, through the anchors on the tooth surface. As mentioned, the oral sensing device may be an oral thermal sensing device.

In some embodiments, the oral sensing device (e.g., oral thermal sensing device) may be configured to be secured to one or more molar teeth. In such devices, the strap may accordingly be dimensioned for molar teeth. Positioning the oral thermal sensing device on the molar teeth may be advantageous in that such region more closely reflects internal temperatures and is less susceptible to variability from the external environment (e.g., temperature loss when the mouth is open) due to its location. Furthermore, the oral thermal sensing device is less intrusive during everyday wear and less visible when placed on the molar teeth.

In some examples, the enclosed sensor unit can be removable from the intraoral device or mount that is worn on the teeth (or a tooth) so that the enclosed sensor unit can be replaced with another unit as needed or modified before recombining the sensor unit with the device or mount. For example, FIG. 5D schematically illustrates an example of an intraoral mount for a removable/replaceable self-locking sensor unit. Removable/replaceable and self-locking sensor units are described in greater detail below in reference to FIGS. 15A-15B and 16A-16B, but may generally include a sensor unit that may be releasably connected to an intraoral device or intraoral mount, an in particular may be configured to self-lock in position. This may allow the same sensor unit to be used with different intraoral devices or intraoral mounts, and/or may allow multiple different sensor units to be used with the same intraoral mount or device.

For example, FIG. 5D illustrates examples of a mount 552 for a removable sensor unit 504′ that may attach and/or be worn on the teeth, and to which a sensor unit 504′ may be coupled. The sensor unit 504′ illustrated in FIG. 5D is configured as a self-locking sensor unit that is configured to couple with an opening in through an intraoral device (in this example, an intraoral mount 552) to be retained securely to the intraoral mount in an assembled configuration, e.g., forming an oral sensing device 550, such as an oral thermal sensing device. The sensor unit 504′ may be separated by removing the assembled oral sensing device 550 and removing the sensor unit 504′ from the back of the intraoral mount. The sensor unit 504′ includes a housing 592 that may at least partially enclose one or more sensors (e.g., a thermal sensor, a pressure sensor, a capacitance sensor, etc.) and a processor that is configured to receive sensor data (e.g., temperature, pressure, capacitance, etc.) from the sensors, and to store, transmit, or store and transmit the sensor data as described herein. The housing includes a flange region at the base 594 of the sensor unit. The flange region may be configured to mate with, and in some examples engage with (e.g., “snap into”) the intraoral device mount 552. In any of these examples the intraoral mount may be more generically referred to as an intraoral device and may include an opening 556 into which the upper region of the housing 592 that is proud of the flange 594 may extend through. This upper region may have an outer diameter that is approximately the same as or less than the inner diameter of the opening 556. The opening may include a receiving seat or lip region around the opening that may engage with the housing of the sensor unit. In some examples the flange and/or channel on the outside of the sensor unit 504′ and the lip region or receiving seat of the intraoral device opening may mate together with a larger tolerance; mating the sensor unit to the intraoral mount (e.g., intraoral device) may secure the two together so that they do not separate when inserting and removing the intraoral device to/from oral cavity and onto the teeth, e.g., similar to what is shown in FIG. 5C. Because the flange is located on the inside of the tooth-receiving region of the intraoral device, when the device is worn, the base 1694 of the sensor unit may prevent the sensor unit from disengaging from the opening of the intraoral device by one or more teeth within the tooth-receiving cavity.

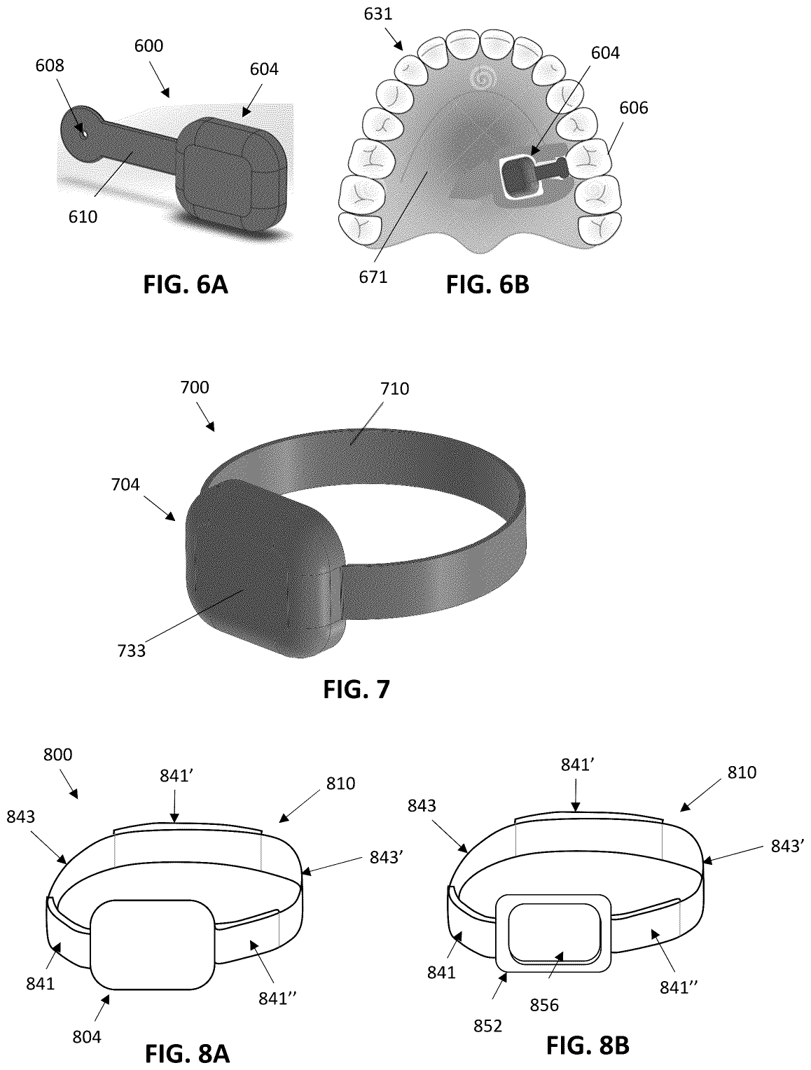

FIGS. 6A-6B illustrate another example of an oral sensing device 600 configured as a stand-alone device. In this example, the oral sensing device also includes a sensor unit 604 and an anchor attachment 608. The anchor attachment may be similar to the anchor attachment in FIGS. 5A-5C including an engagement channel or opening into which an anchor may be removably inserted. In the example device shown in FIG. 6A the anchor attachment is coupled to a strap 610 that is coupled to the sensor unit. In some examples the strap may be relatively stiff, to hold the sensor unit against or near the target region of the oral cavity when the anchor attachment is engaged with an anchor in the oral cavity, as shown in FIG. 6B. in this example the device is secured in position against the palatal region 671. In other examples the strap can configured to be shapable or moldable by hand; thus, for each individual anatomy, the strap can be manually shaped to fit into the space inside the mouth which may allow it to fit better and be more comfortable to wear.

Also described herein are apparatuses in which the oral sensing device includes an elastically adjustable strap to secure the attached sensor unit to one or more teeth. An example of this configuration is shown in FIG. 7. In this example the strap is configured to attach to one or more teeth (although FIG. 7 illustrates a strap around one tooth, the disclosure contemplates that a similar strap may be extended around multiple teeth, e.g., adjacent teeth). The strap is configured as a flexible ring or band, which can be wrapped around a tooth (or teeth) for secure placement of the device. As shown in FIG. 7, the oral sensing device 700 includes a strap 710 that is configured to attach to a tooth (or in some examples two teeth, or more than two teeth). The strap is formed of an elastic material (e.g., a material that is elastically deformable) and the strap is elastically adjustable. This may include all or a portion of the strap. A sensor unit 704 (which may include a thermal sensor, power source, communication circuit, and one or more controllers, not shown) may be coupled to the strap and may include a smooth (atraumatic) outer surface 733. In FIG. 7, a subject wearing the oral sensing device may apply the device by manually attaching it over the one or more teeth so that the sensor unit is facing the proper orientation and/or against the check, gingiva, etc. As used herein the strap may be referred to as an elastically adjustable strap, as it may be elastically deformed by stretching the strap over the one or more teeth (by the action of the subject or caregiver applying the device) such that the clastic strap elastically wraps around the tooth and thus holds the sensor unit against the tooth.

In some examples the strap may be configured to fit between the teeth in some regions and may include one or more regions around the circumference of the strap. The strap may have different regions with different properties. This is illustrated in FIG. 8A. At least one of these regions may be elastic. In the example illustrated in FIG. 8A, the oral sensing device 800 includes a strap 810 that is elastically adjustable and a sensor unit 804 similar to that shown in FIG. 7. The strap 810 includes first regions 843, 843′ that are relatively thin and second regions 841, 841′ that are relatively thick. In some examples, the first region(s) 843, 843′ may be configured to fit between the teeth (e.g., interproximally or interdentally), and the second regions 841, 841 may be configured to engage the buccal or lingual side of a tooth. In these examples, the first regions are made thinner so as to facilitate placement in the tight interproximal/interdental space. In some examples the thickness of the strap in at least these first regions 843, 843′ is 0.5 mm or less (e.g., 0.4 mm or less, 0.3 mm or less, 0.2 mm or less, 0.1 mm or less, etc.), for example 0.2 mm or less. In some examples, the first regions may be relatively less elastic (e.g., more rigid) than the second regions 841, 841′, 841″. Thus, in use, the strap may be elastically expanded from the more elastic first regions while the second regions that are configured to fit between adjacent teeth are strong and relatively less elastic. This may be advantageous in that stronger, more rigid materials may be used for the first regions, which may undergo more frictional stress or wear from adjacent teeth in the interproximal/interdental space. The different regions may be formed of different materials, including different polymeric materials.

As mentioned in reference to FIG. 5D, above, any of these devices may be configured for use with a removable sensor that is configured to removably engage with an intraoral device or intraoral mount. In some cases the removable sensor may be self-locking, e.g., may secured from the back of the intraoral mount or device through an opening. For example FIG. 8B shows another variation in which the sensor unit (not shown) may couple to the intraoral mount 852 through an opening 856 similar to the example shown in FIG. 5D.

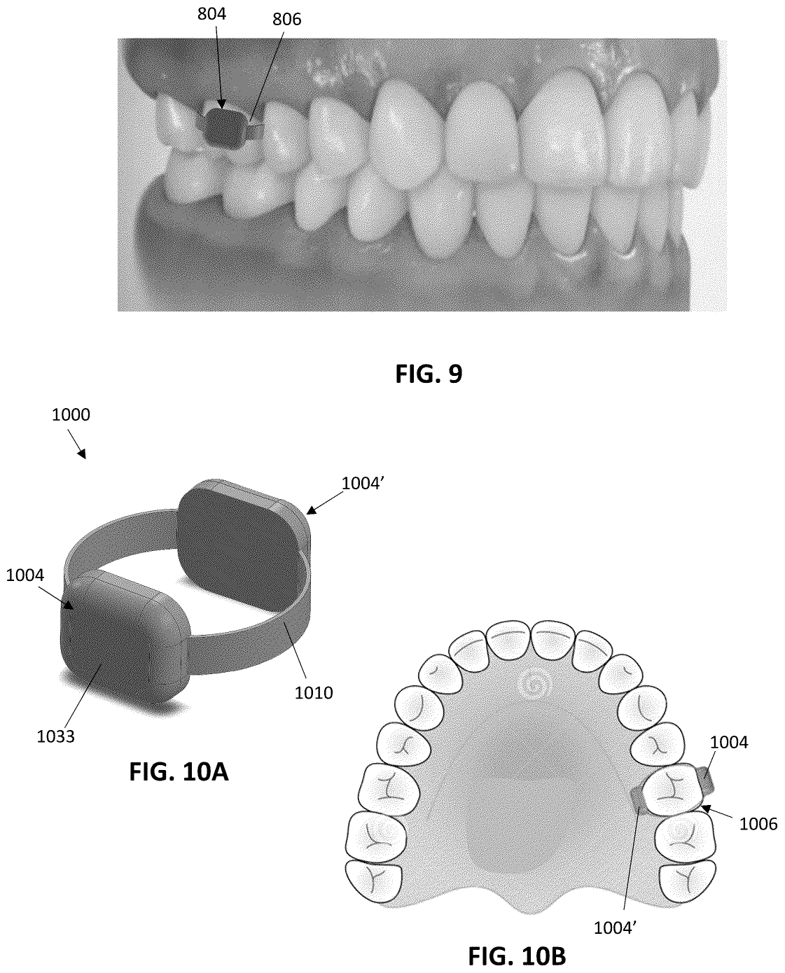

FIG. 9 illustrates an example of a device such as the one shown in FIG. 7 attached to a physical model of a subject's tooth. In this example the sensor unit 804 of the device is held against the buccal side of the teeth in the upper jaw, in a mid-posterior region so that the sensor unit 804 may be held against the check when worn for temperature measurement.

FIG. 10A illustrates an example of a device having two sensor units 1004, 1004′ on a single device. One sensor 1004 can be positioned on the buccal surface of the tooth when the device is worn (e.g., connected by a strap 1010) on one or more teeth. The second sensor unit 1004′ can be positioned on the lingual surface of the one or more teeth. The outside of the sensor units is smooth 1033 and atraumatic. In the examples shown, each sensor unit may be independent, or they may be coupled together either by a wired and/or wireless connection. In some examples the two sensor units may share components, including one or more processors, power supply, etc., or they may be independent. Similarly, multiple sensor units may be positioned on different teeth, and may share or may use different mounts (e.g., bands) securing them in the oral cavity.

As shown in FIG. 10B, the first sensor unit 1004 positioned on the buccal side may be placed in between a tooth 1006 and a check region (not shown) which can provide a stable reference temperature. The second sensor unit 1004′ on the lingual surface may be facing the airway, and the temperature may fluctuate up and down depending on the airflow in the mouth or the temperature environment (indoor vs. outdoor, cold weather vs. hot weather). For instance, this apparatus may detect how much air flows through, so one application of this apparatus may be to monitor the intensity of the exercise by monitoring the user's breathing through mouth while exercising. Also these apparatuses may be configured to monitor sleep quality whether the user is breathing through nose vs. mouth. In some examples snoring or sleep apnea can be detected by monitoring the difference in the temperature between two sensor units. In some examples, the second sensor unit can instead be a power source to power the first sensor unit, so the size of the first sensor unit can be further reduced by having a power source (e.g., battery) that is separated from the primary housing of the sensor unit. In FIGS. 10A-10B, the mount is a flexible band that can include (e.g., may be embedded with) a circuit to connect between the first sensor unit and the second sensor unit (or battery). Alternatively, one of the sensor units can be replaced with another type of sensor unit including one or more different or additional sensors, such as a pressure sensor or a pH sensor, etc. which may complement the temperature sensor and may measure other biometrics (e.g., vital signs and physiological signals).

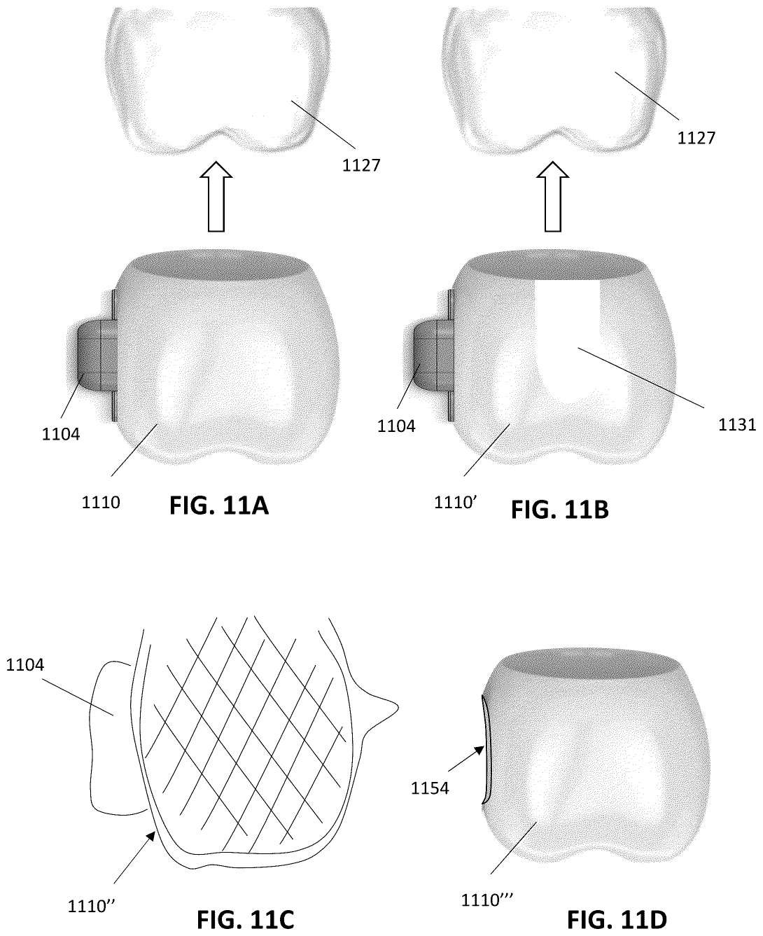

In some examples the mount is configured as a pocket or cap that may fit over one or more individual teeth. For example, FIGS. 11A-11C illustrate examples of sensor units 1104 that are coupled to mounts configured to elastically fit over a subject's tooth (or in some examples, multiple teeth). In the example shown in FIG. 11A, the mount 1110 is an elastic pocket or cap that is adapted to fit over the subject's teeth. The mount may be configured to fit over the tooth in a predefined orientation; for example, the pocket may include regions configured (or customized) to fit over the subject's tooth 1127 or teeth so that the sensor unit 1104 is on a predetermined location and/or orientation (e.g., buccal side, lingual side, adjacent gingiva, check, etc.). In FIG. 11A the pocket or cap is configured to fit onto a tooth. In some examples the tooth may be generic (e.g., not customized) and the pocket or cap may be configured to elastically confirm and fit a variety of different size and shapes. In any of these examples the pocket or cap can be made of an elastic material for secure fitting. In some examples, as shown in FIG. 11C, the pocket or cap 1110″ can be made with a mesh-like configuration that may stretch to fit over the tooth. In some examples, at least a portion of the pocket or cap may be made out of less elastic (e.g., harder) materials such as a metal, hard plastic, etc. Alternatively, the pocket or cap 1110′ may have a precision cut area 1131 (cut-away feature), so it can be fit into a tooth without interfering with neighboring teeth, as shown in the example of FIG. 11B.

In any of these examples the sensor unit may be removably attached to the introral mount, including an intraoral mount configured as a pocket or cap 1110′″ as described above. In FIG. 11D the intraoral mount 1110′″ includes an attachment region configured as an opening 1154 that may engage with removable/replaceable sensor unit, similar to that shown above in FIGS. 5D and 8B. in this example the sensor unit may be self-locking, and may include a base flange that may engage with and/or be retained by an edge or lip region around the inside of the opening 1154 of the introral mount 1110″. Thus, the sensor unit may be inserted from within the inside of the intraoral mount (e.g., pocket or cap 1110′″) and inserted over the tooth to secure the sensor unit within the opening through the intraoral mount.

In some examples, the intraoral thermal data is transmitted when the oral sensing device is not being worn. For example, an oral sensing device worn by a subject may collect and store intraoral temperature data and associated timestamp data for an extended period of time. This stored data may be transmitted to a computing device as described herein (e.g., a smartphone, a laptop/desktop computer). In some embodiments, the oral sensing device (e.g., oral thermal sensing device) may transmit the data directly to the computing device (e.g., via NFC, RFID, Bluetooth, WiFi, a physical cable connection). In some embodiments, a bridging device may be used. For example, the oral sensing device may be placed within a threshold proximity of a bridging device configured as a case/receptacle for the oral sensing device. The data stored within the oral sensing device may then be transmitted to the bridging device via any suitable communication mode. In some cases a low-power communication mode such as NFC or RFID may be used for this transmission to the bridging device. This may allow for conservation of power on the oral sensing device and may further allow for a lower profile of the device (e.g., NFC/RFID circuitry may be less bulky than Bluetooth or WiFi circuitry). The bridging device may then transmit the data to the computing device via any suitable communication mode, including Bluetooth, WiFi, and/or a physical cable.

In some examples, the thermal data may be transmitted to a server (e.g., a remote server) for analysis. In some embodiments, the determination of conditions may be performed at the server. The server may also store the underlying thermal data.

In some examples, the oral sensing devices may be removably attached to an intraoral device. The intraoral device may be one (or a series) of appliance that are configured to be worn on the subject's teeth and may exert a force or forces to control the position and/or orientation of the teeth, or in some cases the palate (e.g., with a palatal expander) or a mandible. For example, FIGS. 15A-15B, 16A-16B, 17A-17B and 18A-18B show examples of a sensor unit that may be releasably connected to an intraoral device. These examples may provide for the use of the same sensor unit across multiple intraoral devices, such as a series of dental aligners used for orthodontic treatment. This may provide cost savings as the number of sensor units needed may be reduced. Removable sensor units may also be advantageous in cases where the associated appliance needs to be cleaned in a manner that would otherwise damage the sensor unit. In some cases it may be particularly beneficial to use the same sensor within the subject's oral cavity with different appliances, as this may avoid the need for calibration. In some cases multiple sensor units may be swapped into the same intraoral device, or the same sensor unit may be removed, modified, and replaced. This may make it easier to change the power supply (e.g., battery), and/or repair the unit. In some cases one unit may be removed to recharge the power source (e.g. battery, capacitor) while another unit may be swapped into the intraoral device.

For example, FIGS. 15A-15B illustrate examples of a removable sensor unit that may be coupled to an intraoral device. This configuration may be referred to as a self-locking design of the sensor unit, as it is configured to couple with an opening in an intraoral device and be retained securely to the intraoral device until it is desired to separate the two. In FIG. 15A the sensor unit 1604 includes a housing 1692 that may at least partially enclose one or more sensors (e.g., a thermal sensor, a pressure sensor, a capacitance sensor) and a processor that is configured to receive sensor data (e.g., temperature, pressure, capacitance) from the sensors, and to store, transmit, or store and transmit the sensor data as described herein. The housing includes a flange region at the base 1694 of the sensor unit. The flange region may be configured to mate with, and in FIGS. 15A and 15B, engage with (e.g., “snap into”) the intraoral device 1682. In any of these examples the intraoral device may include an opening into which the region of the housing proud of the flange may extend through. This region may have an outer diameter that is approximately the same as or less than the inner diameter of the opening. The opening may include a receiving seat or lip region 1683 around the opening that may engage with the housing of the sensor unit. In FIG. 15A the opening on the intraoral device including a lip region 1683 that snaps into a channel on an upper surface of the flange 1681 to secure the sensor unit to the intraoral device. In this case, the surfaces of these regions (e.g., the flange and/or channel on the outside of the sensor unit and the lip region or receiving seat of the intraoral device) may be configured to seal against each other when the two are coupled together as shown.

Alternatively in FIG. 15B the flange 1681 and/or channel on the outside of the sensor unit and the lip region 1683 or receiving seat of the intraoral device opening may mate together with a larger tolerance, as shown. In practice, in either FIGS. 15A-15B, mating the sensor unit to the intraoral device may secure the two together so that they do not separate when inserting and removing the intraoral device to/from oral cavity and onto the teeth. Because the flange is located on the inside of the tooth-receiving region of the intraoral device, when the device is worn, the base 1694 of the sensor unit may prevent the sensor unit from disengaging from the opening of the intraoral device by one or more teeth within the tooth-receiving cavity. Thus, the flange region may be oversized as compared with the opening (e.g., having a diameter that is greater than the diameter of the opening).

FIGS. 16A-16B illustrate one example of a sensor unit 1604 including a flange 1681 on the base of the housing of the sensor unit engaging with an opening 1685 of an intraoral device, configured as an aligner 1602 in this example. A tooth-receiving region 1698 of the aligner is shown in FIG. 16A, and the opening 1685 through the lateral side (e.g., in this example, a buccal side) includes an inner lip region 1683 that may engage with the upper surface of the flange 1681 or a channel (not shown), as described in FIGS. 15A-15B. In use, the sensor unit may be inserted (as illustrated by arrow 1684) from the tooth-receiving cavity 1698 so that it extends out of the aligner 1602, as shown in FIG. 16B. In this example a tooth 1686 is shown within the tooth-receiving region of the aligner, and the self-locking sensor unit is held in place and prevented from disengaging with the intraoral device by the tooth. As shown in this example, the base 1694 of the sensor unit is flush with (or approximately flush with, and in some cases may be slightly recessed relative to) the inner surface of the tooth-receiving region. This may prevent the sensor unit from exerting unintended forces on the teeth.

Thus, the sensor unit may be removed and replaced by removing the intraoral device from the mouth and applying force (e.g., pushing in towards the tooth-receiving region on the sensor unit) to disengage the sensor unit from the intraoral device. The intraoral device shown in FIGS. 16A-16B is an aligner; any appropriate intraoral device may be used, and may be formed from a polymeric material. In some cases the intraoral appliance is a thermoformed material; alternatively the intraoral appliance is formed (including the opening for coupling to the sensor unit) by a direct fabrication technique (e.g., 3D printing).

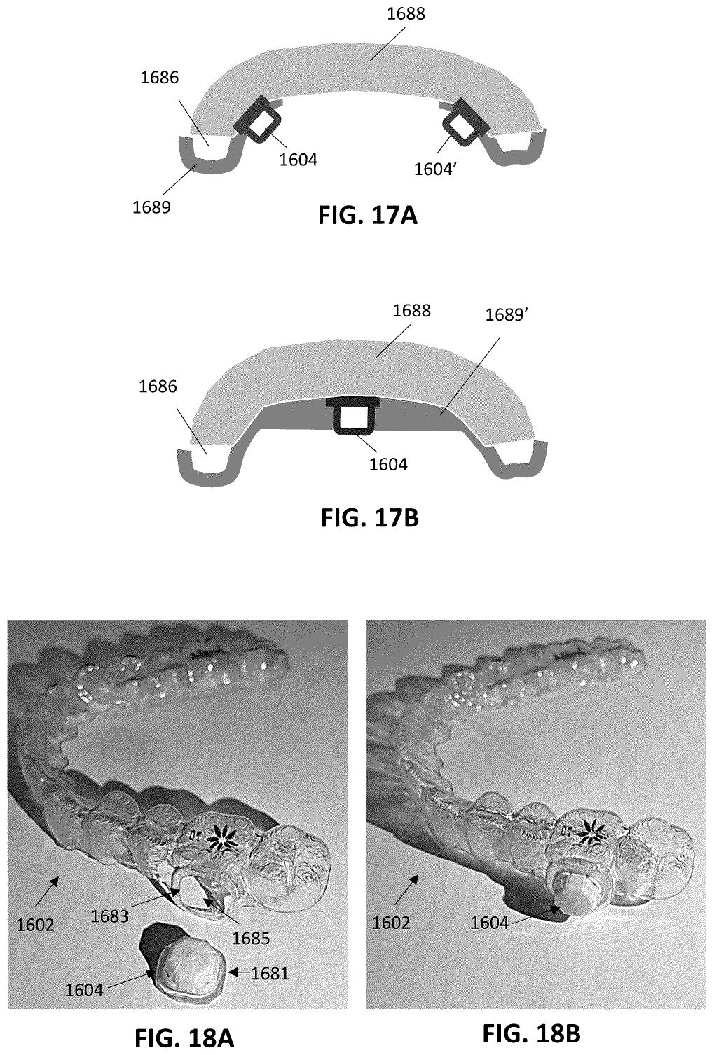

FIGS. 17A-17B show examples in which the intraoral device is a retainer 1689 and a palatal expander 1689′, respectively. These intraoral devices may otherwise be very similar to the aligner shown in FIGS. 16A-16B. In FIG. 17A the intraoral device (retainer 1689) includes a tooth-receiving region holding a tooth 1686 and includes a region that extends from the lingual side towards the subject's palate 1688. In FIG. 17A the apparatus includes a pair of sensor units 1604, 1604′ that are held within opening on the lingual side of the intraoral device. In FIG. 17B the intraoral device is a palatal expander 1689′ that also includes a tooth-receiving region on either side, shown engaged with teeth 1686. In this example the sensor unit 1604 is engaged with and extend through the palatal region of the device. The opening through the intraoral device for holding the sensor unit is located on the central region of the palatal region of the intraoral device. When the palatal expander is worn by the subject the base of the sensor unit may be flush with the palatal region and the sensor unit is prevented from separating form the intraoral device by the flange and the orientation of the sensor unit relative to the palate (e.g., the sensor unit flange is sandwiched between the palatal region of the intraoral device and the subject's palate). Although the sensor units are depicted as being positioned in particular regions within the figures herein, the disclosure contemplates that the sensor units may be positioned in any suitable location. For example, a sensor unit of an aligner may be positioned on a lingual side (similar to the retainer in FIG. 17A). Similarly, a sensor unit of a retainer may be positioned on a buccal side (similar to the aligner of FIGS. 16A-16B). Likewise, a sensor unit of a palatal expander may be on a lingual or buccal side (alternatively or in addition to the palatal region).

FIGS. 18A-18B illustrate an example of an aligner 1602 shown before (FIG. 18A) and after (FIG. 18B) insertion of the sensor unit 1604 including a housing and flange region 1681 around the base (not visible) of the housing. The aligner 1604 includes an opening 1685 having a lip or rim region 1683 that is slightly recessed on the inside region of the opening (e.g., the tooth-receiving side of the aligner).

In addition to the self-locking removable sensor units described above, which may mount through an opening in the intraoral mount (e.g., intraoral device, such as an aligner, palatal expander, etc.) from within the intraoral mount, any of the apparatuses and methods described herein may include removable or replaceable sensor units that may couple to the intraoral mount from the outside of the intraoral mount, or from both the outside and inside of the intraoral mount (or intraoral device). These apparatuses may couple the sensor unit (e.g., thermal sensor unit) to the intraoral mount mechanically, without requiring a bonding material between the sensor unit and the intraoral mount/intraoral device. Thus, these apparatuses may be suitable for large-scale manufacturing and may allow the sensor unit to be removed and/or replaced. This modular design may therefore allow the smee sensor unit to be reusable for multiple times with different intraoral mounts/intraoral devices.

The sensor units described herein may be configured as an electronic module that can be configured with one or more sensors to detect one or more of physical, chemical, and/or biological changes in real-time (including in temperature) that occurs in intraoral space. For example a sensor unit may include one or more sensors to detect force levels, light levels, acceleration, spatial orientation, oxygen saturation, pH levels, and/or sound. The sensor unit can also be configured to transmit and/or store data. The data collected can be used for compliance monitoring, health monitoring, daily activity tracking, sleep quality tracking, appliance locating, dental treatment, and oral health monitoring.

In some examples the sensor units described herein may interlock into a specific compartment of the intraoral mount (e.g., intraoral device), so that the sensor unit can be interchangeable with different intraoral mounts which may be configured to couple to the sensor unit, and in some cases may contain the same compartment. In some cases different compartments in an intraoral mount may hold different components of the sensor unit. For example, the sensor unit may be interchangeable but not a power supply (e.g., battery), and the power supply may be sealed within the intraoral mount. In some examples the sensor of the sensor unit may be embedded but not the data storage unit.

Any of the sensor units described herein may be coupled to an intraoral mount (including but not limited to intraoral devices such as aligners, palatal expanders, etc.) by one or more mechanical locking mechanisms. A sensor unit can be securely attached to an intraoral mount (e.g., intraoral appliance) and it can be configured to be detached and reused multiple times. In any of these examples the sensor unit may be sealed to prevent contamination, e.g., by bodily fluids.

In some examples the sensor unit housing may include a mating component, e.g., a male component and/or a female component, which is configured to secure the component to the intraoral mount (e.g., intraoral appliance). For example, the mating component may be a guide ring, protrusion, threaded projection, etc. The mating component may be integrally formed as part of the intraoral mount, e.g., by 3D printing, injection molding, etc. The mating component may be separately formed and integrated into the intraoral mount. Different materials can be used for the mating component on sensor unit and/or on the intraoral mount. For instance, a metal structure can be used for a male component to provide sufficient strength for secure mounting in a small form factor to minimize overall assembly size.

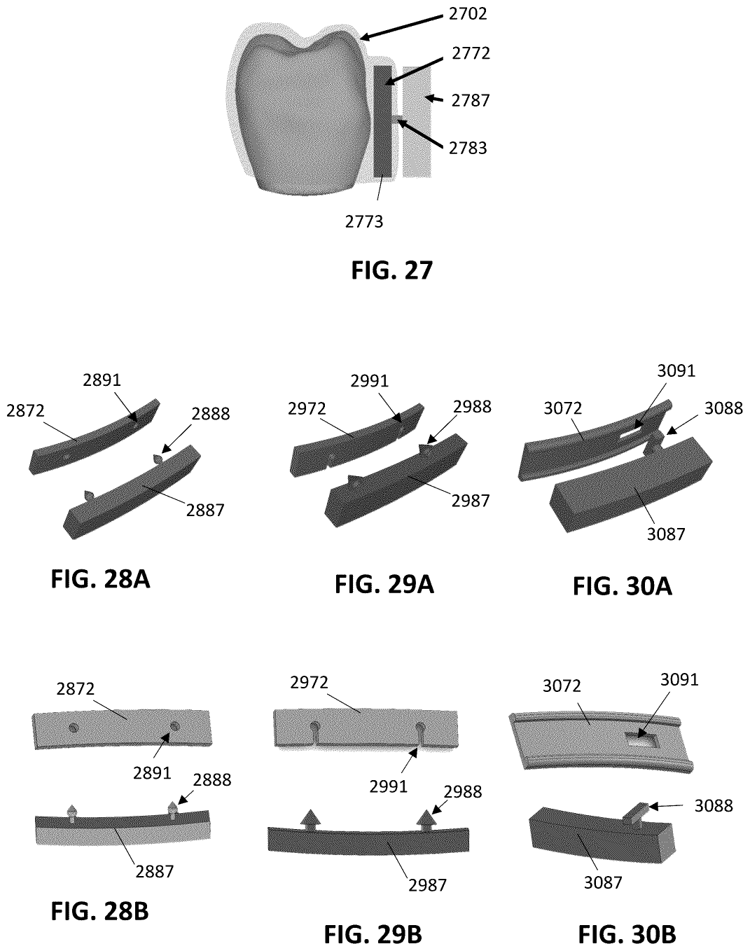

FIGS. 27, 28A-28B, 29A-29B, 30A-30B and 31A-31C illustrate examples of mating components that may be used to couple a sensor unit to an intraoral mount. For example, FIG. 27 shows an example of an intraoral mount 2772 that includes a pocket 2773 holding the intraoral mount; the intraoral mount includes a mating component that includes a well or aperture (e.g., a female pin receiving aperture) that may receive a complimentary mating component on the sensor unit 2787, such a protrusion or pin 2783. In some examples the intraoral mount 2773 on the sensor unit 2787. The sensor unit 2787 may couple to the intraoral mount 2702 by inserting the pin 2783 extending from the sensor unit into the aperture 2773 in the intraoral mount. In some examples the two mating components may lock together when engaged with each other; the lock may be a releasable lock.

For example, FIGS. 28A-28B and 29A-29B show examples of mating components that releasably engage with each other. In FIG. 28A the mating components are configured as snap buttons. The sensor unit 2887 includes one or more (two are shown in FIGS. 28A-28B) posts 2888 having a necked region that may snap into a well or aperture 2891 in the intraoral mount 2872 to secure the sensor unit to the teeth by securing engaging the mating components between the sensor unit and the intraoral mount. The intraoral mount may be integrated with or coupled to (e.g., held within a pocket of) an orthodontic appliance and may be referred to as an intraoral device. In another embodiment, the intraoral mount may be built into an orthodontic appliance without needing a separate part. The intraoral mount may be a portion of the orthodontic appliance with a thicker area including a well or aperture as a part of the orthodontic appliance.

FIGS. 29A-29B illustrate views of another example of a sensor unit 2987 and a portion of an intraoral mount 2972 that may releasably engage with each other through mating components; in FIGS. 29A-29B the mating components include a post having a tapered distal head region and a more proximal neck (similar to that shown in FIGS. 28A-28B) but may engage with a slot 2991 on the intraoral mount 2972. In FIGS. 29A-29B the slot or channel of the mating component may engage with the post 2988 so that the head of the post (pin) 2988 is guided to a well portion that allow the post to lock relative to the intraoral mount. In general, in any of the apparatuses described herein the mating components may be switched between the sensor unit and the intraoral mount. For example, a male mating component (e.g., post, pin, etc.) may be on the sensor unit side and the female mating component (e.g., well, channel, etc.) may be on the intraoral mount side, as shown in FIGS. 28A and 28B. Alternatively the male mating component may be on the intraoral mount side and the female mating component may be on the sensor unit side.

In FIGS. 30A-30B the sensor unit 3087 and the intraoral mount 3072 may releasably engage with each other through mating components configured to operate as a rotation lock. In this example the pin 3088 on the sensor unit 3087 is configured to have an insertion profile that has regions of different diameters, so that when inserted into an aperture 3091 on the intraoral mount 3072, having a complimentary receiving profile, the pin may be rotate so that there is a mis-match between the insertion profile and the receiving profile, locking the sensor unit in position unless the sensor unit and/or intraoral mount are rotated relative to each other so that the receiving profile aligns with the insertion profile. In FIGS. 30A-30B the insertion and receiving profile are rectangular, but other profiles (e.g., triangular, oval, irregular, etc.) may be used.

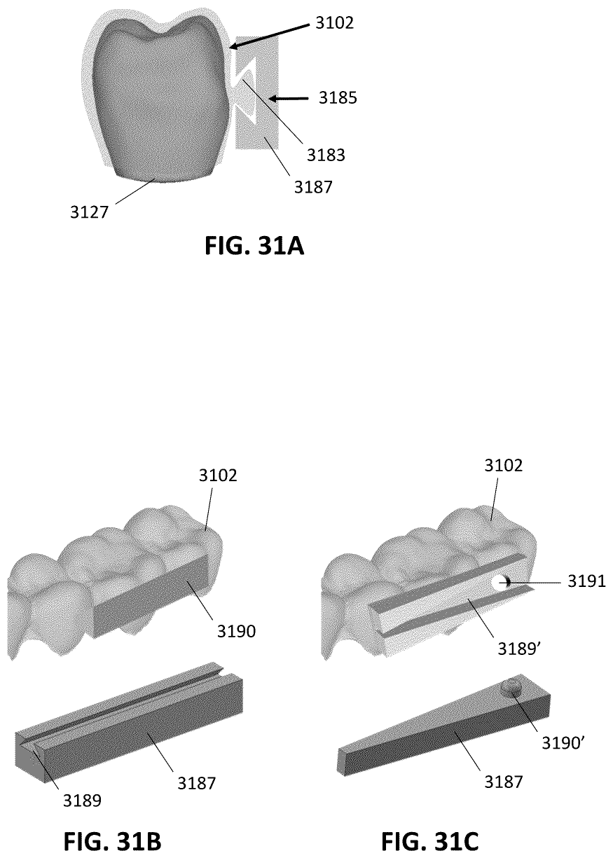

In FIG. 31A the intraoral mount is an intraoral device configured as an orthodontic appliance 3102, e.g., aligner, configured to be worn over the teeth 3127. The intraoral mount includes a mating component comprising a male connector 3183 (e.g., a protrusion having undercut regions) that is shown integrally formed on an external surface of the intraoral device. This male connector may mate with a receiving channel and/or well (female receiving channel or female receiving well) 3185 on an inner surface of the sensor unit 3187.

FIGS. 31B and 31C show alternative examples of configurations of mating components having the same general cross-sectional profile as shown in FIG. 31A. In FIG. 31B the female mating component is a slot or channel (e.g., a trapezoidal slot) within which the male connector (shown as an undercut protrusion) may slide. FIG. 31C shows an example in which the female mating component 3189 is a tapered slot or channel having an opening or aperture. The corresponding male connector 3190 is shown as a triangular prism with a protrusion having an undercut region, may slide until the protrusion is aligned with and engages into a well or aperture 3196 on the female mating component. In FIG. 31B the male mating component 3190 is on the intraoral mount 3102 (e.g., shown as part of an intraoral device, such as an aligner) and the female mating component 3189 is on or part of the sensor unit 3187; this orientation is reversed in the example shown in FIG. 31C. In FIG. 31C the male mating component 3190′ is on, or is part of, the sensor unit 3187, and the female mating component 3189′ is on, or is part of, the intraoral mount 3102 (e.g., shown as part of an intraoral device, such as an aligner). Although particular shapes (e.g., triangular prisms) and features (e.g., undercut) are described for illustrative purposes, the disclosure contemplates any suitable shapes and any suitable features.

Any of the apparatuses described herein may include mating components that are threaded and engage with each other in a screw-on/screw-off manner.

In some examples the mating components may include an active communication, such as an electrical connection. For example, the intraoral mount may include a power supply (e.g., battery, etc.), memory, processor, etc.

### Applications

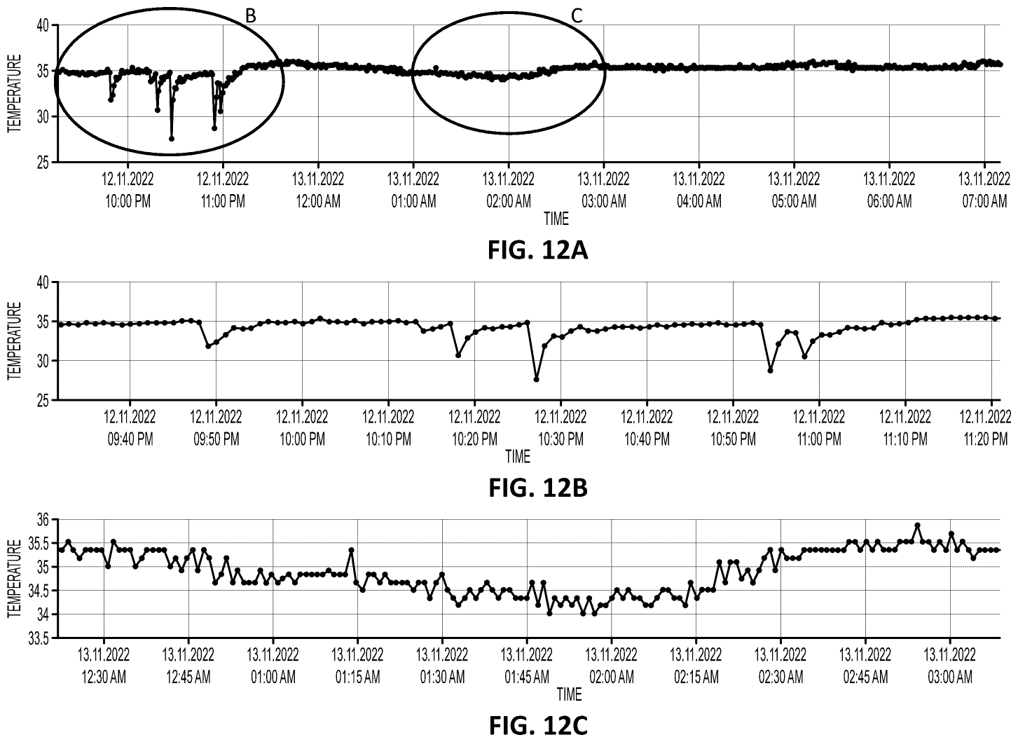

The apparatuses described herein may generally be used to monitor the temperature of the subject. These apparatuses may be configured to measure intraoral temperature and/or may be adapted to measure core body temperature. For example, a prototype device similar to that shown in FIG. 1 was used to measure intraoral body temperature in real time. FIG. 12A is a graph of one example of a period of measurement taken over a ten-hour period during which the device was worn continuously, and temperature data was collected every minute. In this experiment the device was worn for over 20 hours. FIG. 12A provide the raw data and shows how the body temperature, as measured by the intraoral thermal sensor device, changes over time. In this example, the thermal sensor of the sensor unit is configured to have a sensitivity of 0.01 degree Celsius. Over the ten-hour period shown, intraoral temperature changed significantly during a period reflecting consumption of food and beverage (region B, shown in greater detail in FIG. 12B), and during a brief period of sleep (region C, shown in greater detail in FIG. 12C). As shown in FIG. 12B the prototype device detected activities such as drinking and eating. In FIG. 12B the drop in temperature is a result of drinking water. Similarly, during a period of sleep, as shown in FIG. 12C, the device observed a change in intraoral body temperature (a decrease in temperature, as shown) during sleep. FIG. 12C shows that the temperature dropped down to 34 deg Celsius during deep sleep, which can be directly related to overall sleep quality. In addition, snoring with the mouth open could also be identified, e.g., by looking at temperature data patterns or changes.

Thus, continuously monitoring intraoral body temperature over time may detect or diagnose certain events or potential illnesses. Examples may include general fitness (e.g., tracking activity and/or health) and diagnosis of illnesses by tracking intraoral and/or core body temperature using the apparatuses described herein.

In general, the methods and apparatuses described herein may be used to detect core body temperature as derived from the intraoral body temperature measurements typically made by oral thermometers. For example, core body temperature may be determined by intraoral temperatures measured against an inside check region, the gingiva, the palate, and/or other areas within the mouth that are relatively insensitive to rapid changes in temperature that may be reflected in other regions (including the “air” or saliva) of the oral cavity. In some cases, the core body temperature may be estimated by adjusting such measured intraoral temperature, effectively “translating” the measured intraoral temperature to a corresponding core body temperature. This translation may involve executing an algorithm that includes applying one or more functions relating the measured intraoral temperature to the core body temperature. In some embodiments, the functions may first be calibrated for the individual subject for enhanced accuracy. In some embodiments, a translation may not be performed, and the intraoral temperatures may be used directly to perform the applications described herein. In indications in which it is beneficial or desirable to detect rapid changes in the intraoral temperature (e.g., to detect consumption of food, open/closing of the subject's mouth, deep or rapid breathing, etc.) the intraoral temperature may be taken from one or more thermal sensors oriented to measure the temperature of more superficial or airway regions, saliva, etc. Any of the methods and apparatuses described herein may detect multiple regions (airway-exposed/saliva regions and isolated gingiva and/or isolate dentine surface) and may compare the two to determine deviations that may include rapid changes due to consumption, respiration, etc. Changes in the baseline measurements for both may be indicative of changes in core body temperature.

For of the applications described herein, an oral thermal sensing device worn by a subject may collect and store intraoral temperature data and associated timestamp data for an extended period of time. In some embodiments, this stored data may be transmitted (directly or via a bridging device) to a computing device (e.g., a smartphone, a laptop/desktop computer) as described herein. The data may then be logged and analyzed (e.g., by the computing device, by a server to which the data is transmitted) in accordance with the applications described herein.

### Sleep and Circadian Rhythm

In humans, core temperature reliably falls about 2 hours prior to sleep onset and the first non-REM episode is more likely to occur at the steepest point of temperature decline. Different types of insomnia symptoms have been associated with abnormalities of the body temperature rhythm. Sleep onset insomnia may be associated with a delayed temperature rhythm. Circadian rhythm may be disrupted in subjects with dementia and sleep-wake disorders. Circadian rhythm of oral temperature has been shown to be irregularly disturbed in 59.0% of the subjects in a dementia group and in only 12.5% of subjects in the control group. Body temperature rhythm disorganization remained even after treatment of underlying behavioral problems. Thus, behavioral disorders such as delirium, agitation or wandering in subjects with severe dementia might be closely related to disrupted biological rhythms of sleep-waking and the autonomic system (body temperature). The methods and apparatuses described herein may therefore be used to diagnose and/or assist in the treatment of sleep-disordered subjects, including those with behavioral problems.

Thus, the apparatuses and methods described herein may be used to monitor a subject during waking and particularly during sleep in order to detect disruption of sleep and/or the duration of various sleep cycles without requiring the extensive and often uncomfortable and disruptive monitoring equipment typically used for sleep monitoring. The apparatuses described herein may be used with a software application for receiving, storing, visualizing, and/or analyzing the thermal data during waking and/or sleeping period to identify issues with the subject's sleep/wake cycle.

### Aging, Health, and Life Longevity

Healthy aging is associated with core body temperatures that are in the lower range of age-related normal values (36.3+/−0.6 C, oral temperature), while subjects with Alzheimer's disease (AD) exhibit core body temperatures above normal values (up to 0.2 C). Healthy older persons' core body temperatures were found to be in the lower range of age-related normal values and, consequently, lower core body temperature represents a biomarker for healthy aging and longevity. In a 25-year follow-up study, older participants with core body temperatures in the lower 50% of the population, had significantly lower mortality than those with core body temperatures in the upper 50%. Furthermore, lower early morning core body temperature (assessed by oral thermometer measurement) is related to better physical fitness, including faster gait speed, reduced 400-m-walk time, and lower perceived exertion after walking 5 min at 0.67 m/s. Thus, the methods and apparatuses described herein may be used to provide an indicator of health and/or aging generally or with respect to specific indications.

For example, the methods described herein may be used to determine, diagnose, or track disorder such as diabetes risk. Subjects who have a “thrifty” phenotype and are at greater risk for type-2 diabetes exhibit lower body temperatures and have certain trending body temperature loss when eating after fasting. The apparatuses described herein may detect both core body temperature as well as consumption of food and beverage, based on detection of the intraoral temperature, as described. Thus, these methods and apparatuses could be used to predict a risk of diabetes.

For example, core body temperature (CBT) increases with feeding and is lower when fasting. To a different degree depending on the macronutrient composition of the overfeeding diet of subjects, there is a correlation between diet and how much CBT increased during feeding. There is a ceiling effect such that individuals with a higher CBT during fasting have limited capacity to increase CBT with feeding. Long term calorie restriction and fasting is associated with lower core body temperature. Compared to endurance exercise, the temperatures were significantly lower in those that had calorie restriction diets than exercising individuals.

A number of performance measures have been shown to be better when body temperature was elevated, including working memory, subjective alertness, visual attention, and the slowest 10% of reaction times. These findings demonstrate that an increased body temperature, associated with and independent of internal biological time, may be correlated with improved performance and alertness. In general, the body may be under the most strain and may perform most optimally at higher temperatures, as it may be primed for activity (mental, physical, etc.). The methods and apparatuses described herein may allow a subject to track periods during which optimal performance may be achieved.

For example, healthier people typically exhibit lower average body temperatures and increased peak-to-peak body temperatures while performing tasks. Alzheimer's subjects have on average increased core body temperatures compared to age-matched controls (e.g., 0.2 degrees C.). When performing mental and cognition tasks, higher peak body temperature while performing the tasks correlated to better performance during the task. Younger individuals typically had lower body temperature. The methods and apparatuses described herein may allow tracking of body temperature long term and may allow checking of peak-to-peak temperature and average temperature as a biomarker for cognitive decline or issues in health. These methods and apparatuses may also allow the subject to perform mental tasks when the body can reach peak core body temperatures for optimum performance.

Thus, the apparatuses and methods described herein may be used to monitor a subject's temperature throughout the day to identify patterns that may be predictive of overall body health or likelihood of diseases. The patterns may be analyzed and visualized on an associated software application, as described herein. The software application may provide conclusions of an analysis in any suitable manner. For example, it may present one or more numerical values that correspond to risk of diabetes, healthy aging, etc.

### Performance, Recovery, and Diet

The methods and apparatuses described herein may also or alternatively be used for the early detection of excessive perturbations of core body temperature, which may also be helpful in monitoring, detecting and/or diagnosing athletic recovery and performance problems, such as heat stroke. For example, these methods may be used to determine if an individual should receive interventional cooling strategies to avoid exertional heat stroke during or after exercise. For example, body temperature reaches a much higher peak during high vs. moderate intensity and remains elevated for longer following recovery, e.g., approximately one degree change from exercise through recovery process. In addition, these methods and apparatuses may be useful to guide a subject in enhancing the timing and performance of exercise, e.g., to optimize performance within different core temperature windows in order to maximize output.

For example, the methods and apparatuses described herein may allow a user to track core body temperature in order to guide training (physical training). For example, heat training may help reduce heat stress and, in turn, improve performance and recovery. When you get hot, your performance drops. Some cyclists train in “heat stress” conditions to get their bodies used to it and condition them to adapt to the heat better.

As described above, intraoral temperature may be used to determine when a user is eating and/or drinking. For example, patterns in temperature changes from such activity can be used to determine when a subject is eating or drinking. This may be tracked in an associated software application as described herein. The software application may present to the subject a visualization that, for example, shows the times during each day that the subject ate or drank. This may be used by the subject, for example, to track compliance to a dietary plan (e.g., fasting, intermittent fasting, calorie restriction) over a period of time. As another example, a subject may use the data to track consumption of water to ensure proper hydration. In some embodiments, the software application may also present recommendations based on the recorded eating and/or drinking times. For example, the software application may push a notification (e.g., on a mobile device) reminding the subject to eat and/or drink. As another example, the software application may push a notification informing the user that eating and/or drinking has been detected, and reminding the user that a dietary plan of the subject does not permit such eating and/or drinking.

### Menstruation and Pregnancy

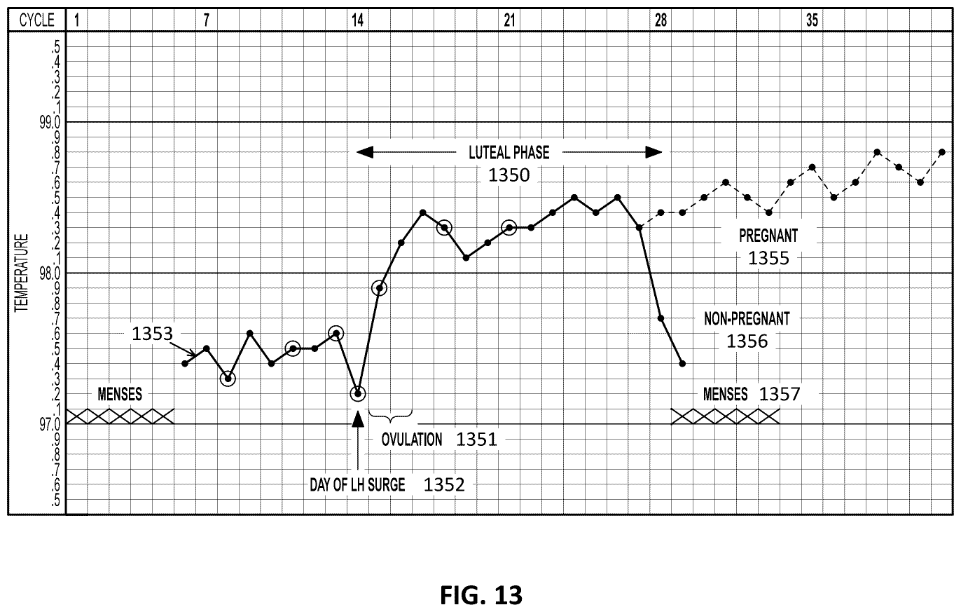

The methods and apparatuses described herein may also be used to track fertility in women. For example, body temperature is typically lower in the first part of a woman's cycle, and then rises when the woman ovulates. For most women, 96°-98° Fahrenheit is a typical temperature before ovulation. After ovulation, body temperature may go up to 97°-99° F., about four-tenths of one degree higher than their usual temperature. As shown in FIG. 13, core body temperature, which may be derived from intraoral temperature, may predict fertility (e.g., from period of menses 1357 to the luteinizing hormone (LH) surge 1352 and ovulation 1351.

Temperature may be sustained relatively high over the luteal phase 1350 and may fall back to the lower level if fertilization does not occur 1356, whereas the temperature may remain relatively elevated in a pregnant person 1355. Thus, the methods and apparatuses described herein may track and detect these different periods of fertility and/or pregnancy.

These methods and apparatuses may also be used to detect, track and/or diagnose problems with female fertility. For example, statistically significant differences have been seen in the temperature curves of women with luteal phase deficiency and polycystic ovary syndrome compared to women with normal menstrual cycles. The analysis of individual cyclofertilograms can be used to detect cycle phases and estimate the date of ovulation.

### COVID and Virus Infection Tracking

The methods and apparatuses described herein may generally be configured to detect, diagnose and/or monitor infection. Infection is generally known to result in changes in body temperature, both core and peripheral body temperature. In addition, these methods and apparatuses may be used to differentiate among different types of infection, including different viral infections (e.g., COVID and Influenza) and/or bacterial infections. For example, it has been shown that when temperature was monitored for over 72 hours, fever (>37.6° ° C. or 99.7° F. or a change in temperature of >1° C. or 1.8° F.) was detected in 73% of COVID-positive individuals, a sensitivity comparable to rapid SARS-COV-2 antigen tests. When compared a control group, the specificity of fever for COVID-19 was 0.70. However, when fever was combined with complaints of loss of taste and smell, difficulty breathing, fatigue, chills, diarrhea, or stuffy nose the odds ratio of having COVID-19 was sufficiently high enough so as to obviate the need to employ RTPCR or antigen testing to screen for and isolate coronavirus infected cases. COVID-19 positive subjects had higher mean body temperature compared to COVID-negative subjects. In contrast, influenza subjects had even higher mean body temperature, heart rate, and oxygen saturations than COVID-19-positive and -negative subjects. Thus, mean body temperature (which may be derived from intraoral temperatures) can be used to differentiate between COVID-19 and influenza.

Thus, the methods and apparatuses described herein may determine changes in core body temperature to provide an early-warning system for subjects of an infection. The extended monitoring capability provided by the described methods and apparatus may allow subjects to proactively determine such an infection and take steps to mitigate the illness (e.g., by drugs or supplements) and/or to prevent spread (e.g., by isolating from others, by wearing personal protective equipment such as masks). Furthermore, the accuracy in temperature monitoring allowed by intraoral measurements (as opposed to other noninvasive methods) allows for differentiating among different infections that cause the body to respond with different temperature patterns. As described above, COVID-19 and influenza have different temperature patterns. By examining thermal data from a subject wearing the described oral thermal sensing device, software may determine that the subject's mean body temperature is within a heightened range (indicating an infection) but that the mean body temperature is below the range for influenza (indicating that the infection is unlikely to be influenza, and may be COVID-19). In this way, any suitable two or more infections that cause different body temperature patterns may be differentiated. In conjunction with subject-reported symptoms, measured intraoral temperatures may be a power predictor for detecting infection, and also for differentiating among different viral infections, including (but not limited to) COVID and Influenza. Although the disclosure focuses on detecting infection and differentiating among infections, the disclosure contemplates such detection and differentiation for any illnesses that affect body temperature patterns.

### User Interface

In general, the method and apparatuses described herein may include software, including application software that may run on a subject's personal computing device, such as a phone, tablet, smartwatch, laptop/desktop computer, etc. that may receive, process, transmit and/or display information temperature readings and/or information extracted from monitoring temperature readings, as described above. The application software may execute on one or more processors of the personal computing device and/or may communicate with another software hosted on a remote (e.g., cloud-based) server for further processing of the temperature reading and/or control of the oral thermal sensing device. In general, the application software may receive data (e.g., temperature readings, timing, changes in temperature, etc.) from the oral thermal sensing device, and/or may transmit control information to adjust the operation of the device, including turning the oral thermal sensing device on/off, and changing the sampling rate, mode, or operation, etc.

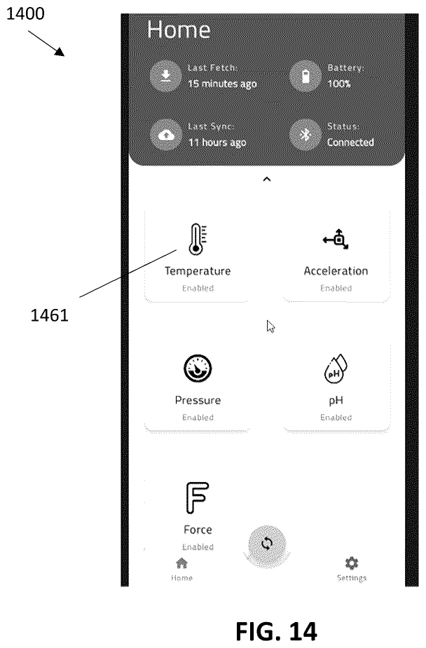

For example, FIG. 14 illustrates one example of a user interface 1200 for an application software that may run on a subject's smartphone. In this example the application software may include status indicators (e.g., last fetch, battery of the oral thermal sensing device, last sync, status, etc.) and may allow monitoring of the sensor unit output. In some examples (as shown in FIG. 14) multiple different sensors may be controlled, tracked, and/or visualized using the same software. The software may also execute one or more methods in which data from multiple different sensors (multiple temperature sensors 1461 and/or one or more temperature sensors and other sensors, such as accelerometers, pressure, pH, force, etc.). These sensors may be part of the sensor unit or may be separate.

The application software may receive, store, transmit and/or display the temperature readings and/or the results of any analysis of the temperature data. For example, the user interface may be configured to display graphical data of temperature data, e.g., FIGS. 12A-12C and 13. The application software may receive user input to change the operation of the oral thermal sensing device (e.g., turning on/off, setting frequency rate, etc.). The user interface may also or alternatively be used to enter additional data, including subject-reported data (general health data, etc.). Finally, the user interface may output detection, diagnostic and/or monitoring data as described herein.

The application software may also provide notifications and/or recommendations (e.g., via push notifications, email, text message) based on the analysis of the received temperatures. For example, the application software, upon determining elevated mean body temperatures within a range corresponding to an infection, may send a notification to the subject that they may have an infection. As another example, the application software may also provide a recommendation (e.g., to get tested, to isolate, to wear personal protective equipment).

### Temperature Sensing

In operation, these apparatuses described herein may be configured to sense temperature within the subject's oral cavity in an ongoing, scheduled, or on-demand manner. In some cases the temperature sensing may be ongoing at a sampling frequency that is constant or variable. Any of the methods and apparatuses described herein may determine if a sensed temperature is reliable or stable, and may keep or discard temperature data, or in some cases modify the sensed temperature data if the data is determined to be unstable. In particular, these methods and apparatuses may determine if sensed temperature is unreliable or unstable in cases where the subject is eating or drinking, which may result in sensing the temperature of the food or beverage being consumed, rather than body temperature; or if the subject's mouth is open, because of excessive breathing; or during exercise, which may result in an unstable or inaccurate temperature reading, particularly if the ambient temperature is much lower or higher than body temperature.

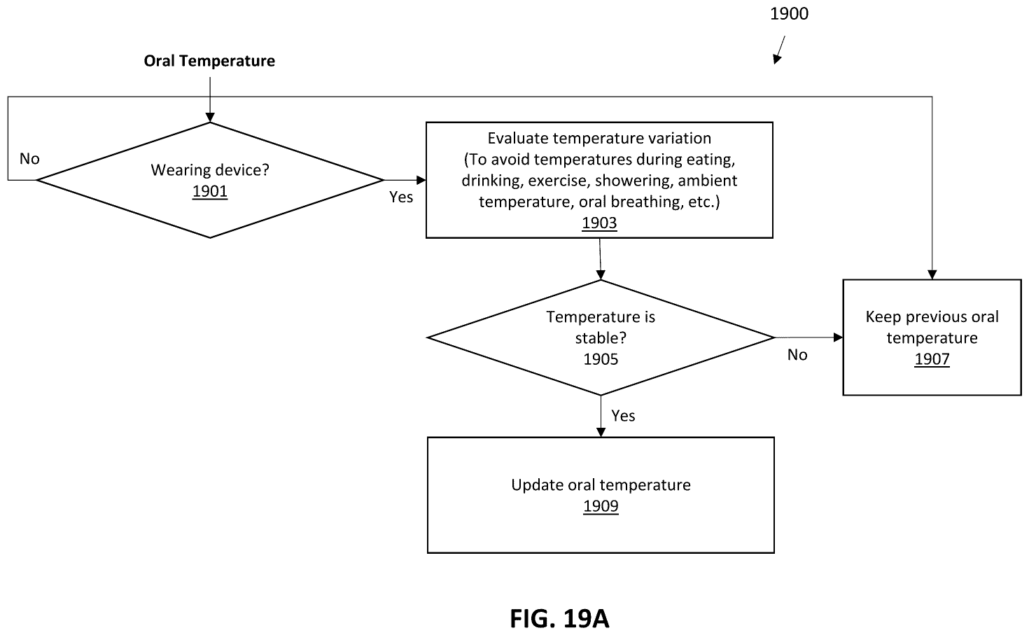

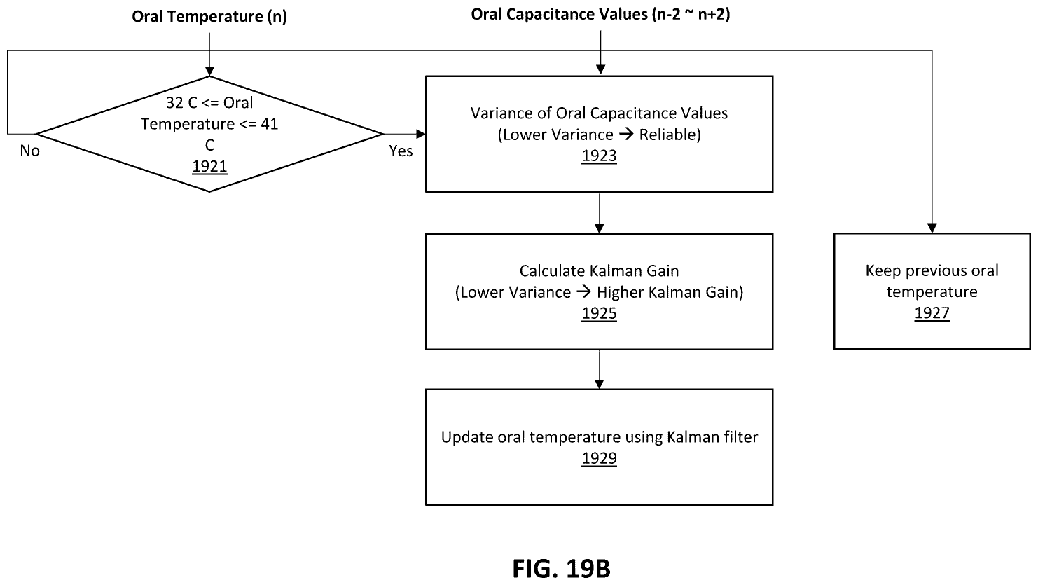

For example, FIGS. 19A-19B illustrate examples of measuring an oral temperature that is representative of body temperature including confirming that the temperature is stable and/or accurate. FIG. 19A shows one example of a method 1900 that receives intraoral thermal data from an oral thermal sensing device worn in the subject's mouth. The method (or an apparatus configured to perform this method) may determine if the subject is wearing the thermal sensing device within the oral cavity 1901. For example, the method may include checking if the subject is wearing the device based on one or more additional sensors on the device, such as an electrical sensor that can detect or confirm contact with the tooth/teeth (e.g., capacitance sensor), or one or more sensors configured to confirm that the device is within an oral cavity environment based on pH and/or another biomarker. In some cases the device may confirm contact based on a mechanical sensor (e.g. pressure/contact sensor).

If the device is not determined to be present in the body based on such data, the value of the received “oral temperature” reading may be discarded and the subject's temperature may be kept as the prior legitimate temperature reading 1907. In some cases, if the temperature is not determined to be reliable, one or more additional readings may be performed and used to determine the actual temperature, and/or the method or apparatus may be configured to trigger an alert indicating that the results are not stable, and the sensor is likely not in contact (or in sufficient contact) with the oral cavity. In some cases multiple repeated readings may be taken until it is confirmed that the subject is wearing the device.

Assuming that the device is being worn, the method may next evaluate the temperature variation of the oral temperature measurement(s) received 1903. For example, a window of time may be observed, within which multiple oral temperature readings may be taken using the thermal sensing device. In some cases the pattern of change of the temperature overt his relatively short duration or window (e.g., approximately 5 seconds, 10 second, 15 seconds, 20 seconds, 25 seconds, 30 seconds, 35 seconds, 40 seconds, 45 seconds, 50 seconds, 60 seconds, 1.25 minutes, 1.5 minutes, 1.75 minutes, 2 minutes, 3 minutes, 4 minutes, 5 minutes, etc., including between 5 seconds and 5 minutes, between 5 seconds and 4 minutes, between 10 seconds and 3 minutes, etc. or any other range of times between these) may be examined to determine if the subject is eating, drinking, showering, etc. The pattern of change of the oral temperature may be indicative of one or more of these states. For example, rapid changes in the temperature within the window of time examined, particularly when centered around an expected body temperature range (or in some cases, a baseline body temperature range) may correspond to eating or drinking a hot or cold food/beverage. Any of these methods and apparatuses may use a trained pattern-recognition agent (e.g., a trained machine learning agent) to identify these patterns and may remove of adjust the oral temperature reading. In FIG. 19A, the method may include determining if the temperature is stable 1905. The stability of the oral temperature received may be determined as mentioned above, and/or may be determined based on the variance of the temperature within the window of time being considered. In some cases the stability may be estimated if the prior step does not recognize a behavior based on the pattern of temperature readings within the time window. If the variance of the temperature over this window of time is outside of an acceptable variance range (threshold) then the oral temperature reading may be rejected, and method may instead use the prior temperature 1907 and/or may trigger a flag indicating an unstable temperature reading. If the temperature appears otherwise stable, the method may update the current oral temperature 1909.

FIG. 19B illustrates another example of a method of determining the oral temperature reading, using the Kalman filter technique to reflect variability of the oral temperature reading(s). For example, in FIG. 19B, the method may include receiving the oral temperature and confirming that the subject is wearing the device, e.g., based on the received temperature being within the expected body temperature range (e.g., between about 32 degrees C. and 41 degrees C.) 1921. If not, the measurement is rejected 1927 (e.g., the prior oral temperature value may be used and/or a flag or alert that the temperature is outside of the allowable range may be triggered). If the temperature from the sensor unit (e.g., the thermal sensing device worn in the subject's mouth) is within the acceptable range of temperature values, the method may use one or more capacitance values, e.g., oral capacitance values from a window before and after the temperature measurement at time n (e.g., n−2 to n+2) and may determine that the valiance of the capacitance values 1923. Generally, the lower the variance, the more reliable the value is. A low variance of the capacitance may mean that the device is held within the mouth (e.g., has not been removed). The Kalman filter may then be applied using the temperature data and variance of capacitance values, e.g., within the same window of time, for time n 1925. Kalman filtering, also known as linear quadratic estimation (LQE), uses a series of measurements observed over time, including statistical noise and other inaccuracies, and produces estimates of unknown variables that tend to be more accurate than those based on a single measurement alone, by estimating a joint probability distribution over the variables for each timeframe. The Kalman gain is the weight given to the measurements and current-state estimate, and can be “tuned” to achieve a particular performance. With a high gain, the filter places more weight on the most recent measurements, and thus conforms to them more responsively. With a low gain, the filter conforms to the model predictions more closely. At the extremes, a high gain (close to one) will result in a jumpier estimated trajectory, while a low gain (close to zero) will smooth out noise but decrease the responsiveness. Although this discussion focuses on the use of a particular filter technique and statistical method, any suitable filters and statistical methods may be used for similar purposes.

As shown in FIG. 19B, the method may include updating the oral temperature using the Kalman filter 1929. Thus, the window of time around the time being examined may be used to determine the final output oral temperature.

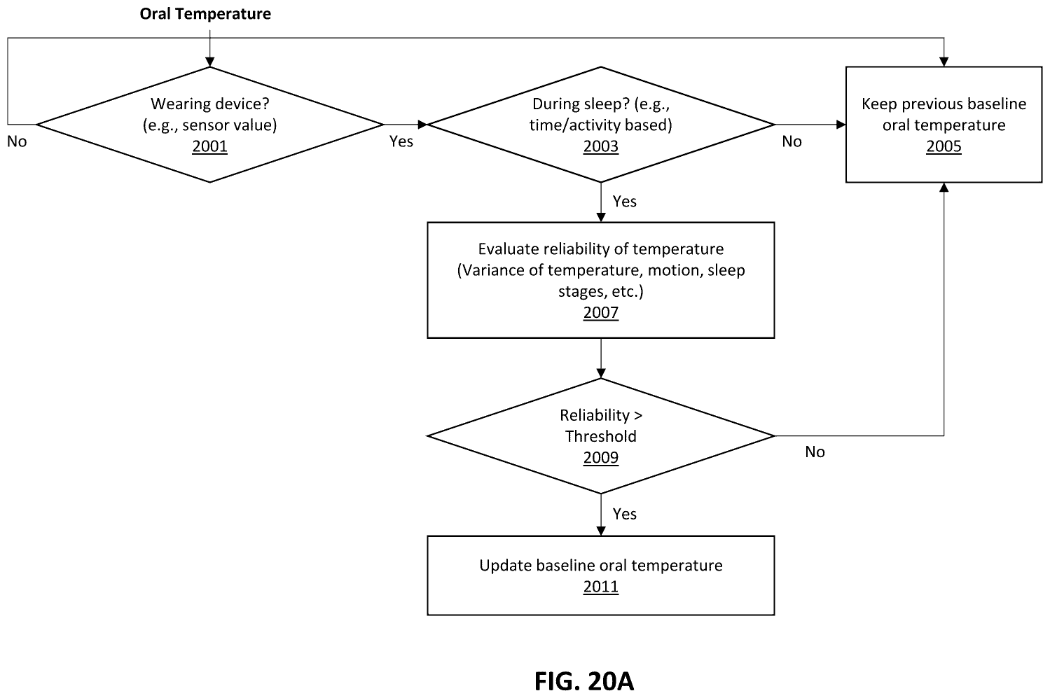

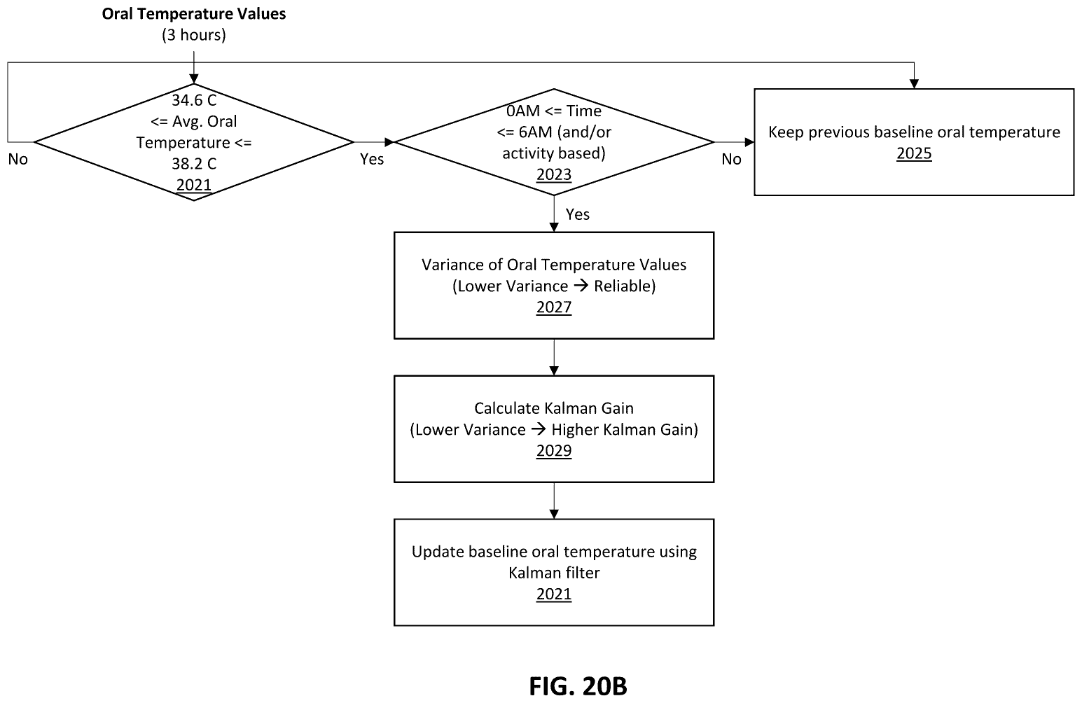

Any of these methods and apparatuses may also estimate a baseline temperature for the thermal sensing device (e.g., sensor unit). FIGS. 20A-20B illustrate examples of methods for determining a baseline temperature specific to the subject. The baseline temperature may be used to determine if the recorded temperature is unusual, and may indicate one or more of the states described above, including detection of elevated temperature (e.g., fever), and/or detection of physical activity or health, e.g., if the temperature is lower than the baseline by a threshold amount.

The baseline oral temperature may be determined for a specific subject and may represent the oral temperature when the subject is at rest, including, e.g., after a period of uninterrupted sleep. Factors that may influence the baseline may include illness, changes in sleep patterns, alcohol consumption, interrupted sleep, travel, certain medicines, etc. As described herein, the baseline oral temperature value may be updated periodically in an ongoing manner.

FIG. 20A shows a first method for determining baseline temperature using a thermal sensing device worn in the subject's mouth. In FIG. 20A the oral temperature is received and, as discussed in reference to FIGS. 19A-19B, the method may confirm that the subject is wearing the thermal sensing device 2001. For example, based on one or more sensors, which may be part of the thermal sensing device. If the device is determined not to be worn by the subject, the method may maintain the current baseline (or may set if to an automatic level) 2005. Otherwise, the method may determine if the subject is within a period of low activity, such as sleeping or repose. The activity level may be determined in some case by the time of day (e.g., between midnight and 5 am), or based on a sensor (e.g., accelerometer or other motion sensor) 2003. If the subject is active or expected to be active, the method may simply maintain the prior (or a preset) baseline temperature value 2005, otherwise the method may include evaluating the reliability of the temperature data received, e.g., by looking at the variance 2007. A low variance for the temperature (e.g., within a window of time before and/or after the temperature reading) may be estimated and compared to a threshold. If the variability is less than the threshold 2009, the method may then update the baseline oral temperature 2011.

FIG. 20B is another example of a method of determining an oral temperature baseline. In this example the method may be taken periodically (e.g., every x hours, such as every hour, every 2 hours, every 3 hours, etc.) and may initially compare the temperature to a range to confirm that the subject is wearing the thermal sensing device worn in their mouth 2021, as described above, such as by comparing the sensed temperature to a range of between 34.6 degrees C. and 38.2 degrees C. (or between 32 degrees C. and 41 degrees C., etc.). If the temperature is within range, the method may determine if the time of day suggests that the subject is asleep (e.g., time is between midnight and 6 am, etc.) 2023. If not, the previous baseline value may be kept (or a default baseline value may be used) 2025. If so, then the method may determine the variance of the oral temperature values, e.g., within a window before/after the oral temperature value 2027. As described above in reference to FIG. 19B, the method may use a Kalman gain estimate 2023 and a Kalman filter 2021 to first determine if the variance is sufficiently low so that the temperature measurement (e.g., within a window of time). The filtered valve may then be used as the baseline oral temperature value.

As mentioned above, the methods and apparatuses described herein may include methods and apparatuses for determining if a subject's body temperature is greater than an expected value (e.g., determining if the subject has a fever). A normal body temperature is ordinarily maintained despite environmental variations because the hypothalamic thermoregulatory center balances the excess heat production derived from metabolic activity in the body. The mean normal oral temperature may be between 36.8°+0.4° C. (e.g., 98.2°+0.7° F.), however this value may change over the course of a day, with low levels at 6 A.M. and higher levels at 4-6 P.M. The maximal normal oral temperature is typically 37.2° C. (98.9° F.) at 6 A.M. and 37.7° C. (99.9° F.) at 4 P.M.; these values define the 99^{th} percentile for healthy individuals. In light of these studies, an A.M. temperature of >37.2° C. (>98.9° F.) or a P.M. temperature of >37.7° C. (>99.9° F.) may define a fever. The normal daily temperature variation, also called the circadian rhythm, is typically 0.5° C. (0.9° F.). However, in some individuals recovering from a febrile illness, this daily variation can be as great as 1.0° C. During a febrile illness, the diurnal variation is usually maintained, but at higher, febrile levels. The daily temperature variation appears to be fixed in early childhood; in contrast, elderly individuals can exhibit a reduced ability to develop fever, with only a modest fever even in severe infections.

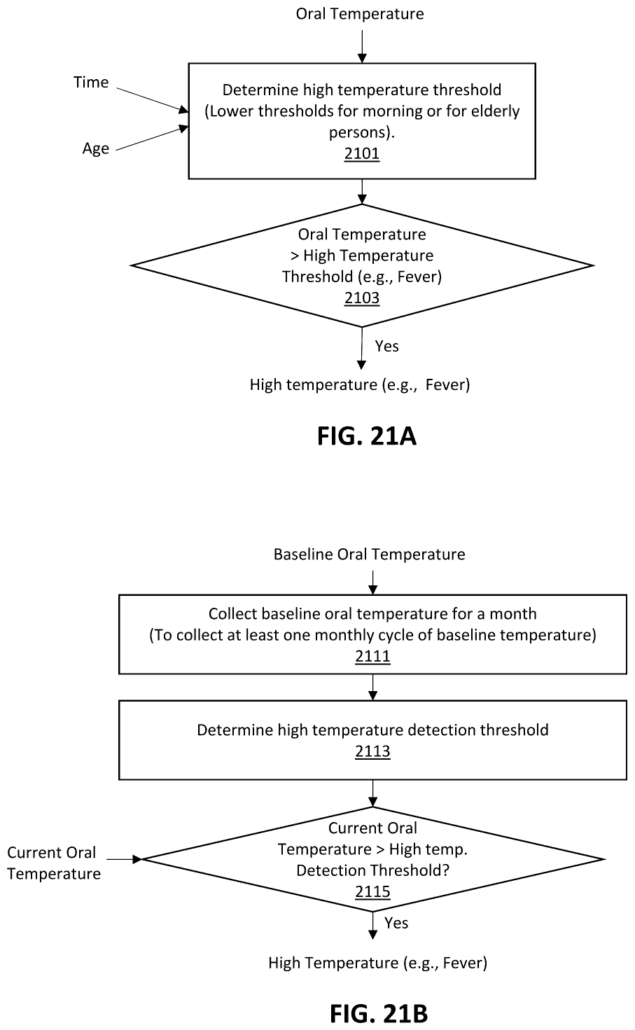

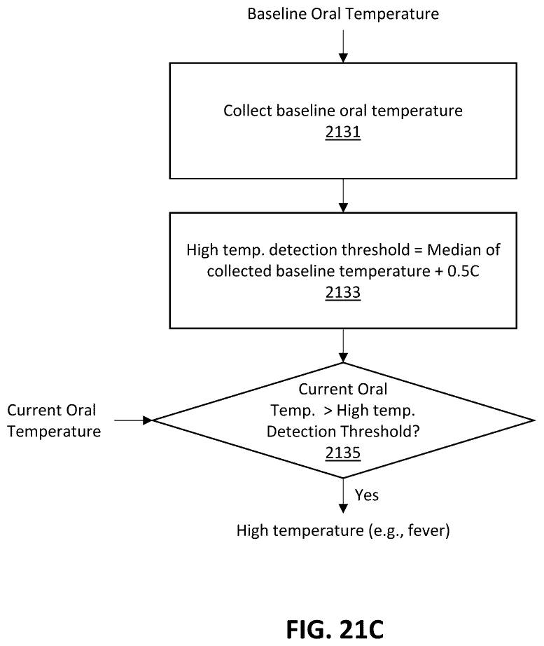

FIG. 21A illustrates one example of a method of determining if a subject has a higher than normal (e.g., feverish) temperature using a thermal sensing device worn in the mouth. In FIG. 21A, the method may include using the sensed oral temperature from the thermal sensing device, as well as the time of day and/or age of the subject, may determine if the oral temperature sensed is greater than an elevated temperature threshold (e.g., a high fever temperature threshold). As mentioned, this threshold may be adjusted based on the subject's age and based on the time of day 2101. If the oral temperature from the sensor unit of the thermal sensing device (or from multiple sensor units) is greater than the elevated temperature threshold 2103, the method or apparatus may indicate that the subject has an elevated temperature (e.g., fever).

FIG. 21B illustrates another example of method of determining if a subject has an elevated temperature using the thermal sensing device. In FIG. 21B, the method may include the use of the subject's baseline temperature. The method may include determining the baseline oral temperature from the thermal sensing device for the particular subject. This may be determined over a period of time, such as a week, month or multiple months. In particular, the method may determine the baseline oral temperature over at least a month, in order to capture the monthly cycle in the temperature 2111. The method may determine if an elevated temperature (e.g., in some cases a high fever) by comparing the measured oral temperature to a threshold that is based on the subject's particular baseline 2113. For example, the elevated temperature detection threshold may be set as an offset (e.g., 0.2 degrees C., 0.3 degrees C., 0.4 degrees C., 0.5 degrees C., 0.6 degrees C., 0.7 degrees C., 0.8 degrees C., 0.9 degrees C., 1.0 degrees C., etc.) of the baseline. The elevated temperature detection threshold (e.g., from the baseline and/or the offset) may be adjusted based on the subject's age and/or time of day, as mentioned above. If the current oral temperature is greater than or equal to the (or in some cases, just greater than) the elevated temperature detection threshold 2115.

FIG. 21C illustrates another example of a method for detecting an elevated temperature in a subject wearing a thermal sensing device as described herein. In FIG. 21C the method may include collecting the baseline oral temperature for the subject 2131. As mentioned above, the baseline oral temperature for the subject may be used to determine a subject-specific elevated temperature (e.g., fever) detection threshold 2133, for example by adding an offset (e.g., 0.2 degrees C., 0.3 degrees C., 0.4 degrees C., 0.5 degrees C., 0.6 degrees C., 0.7 degrees C., 0.8 degrees C., 0.9 degrees C., 1.0 degrees C., etc.). The method may determine if the subject's current oral temperature is greater than the elevated temperature detection threshold 2135. If so, the method may indicate that the subject has an elevated temperature or not. In some cases if the fever is detected, the method and/or apparatus may indicate that an elevated temperature is detected by emitting an alert, including transmitting to a remote device.

### EXAMPLES

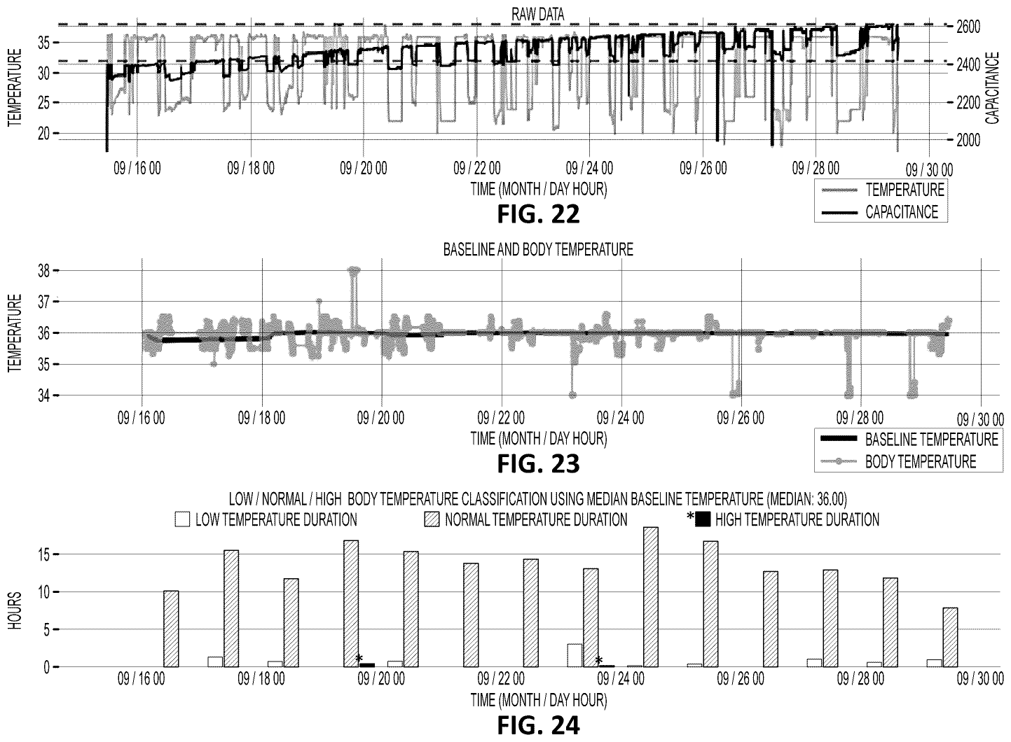

FIGS. 22-26 show examples of a data collected using thermal sensing devices including a sensor units as described above. Subjects wore these apparatuses for one or more days, typically as a subject-removal appliance, such as an aligner, to which the sensor unit was attached, such as is shown in FIGS. 1 and 3. The temperature data was collected as described above. In some cases the temperature data was checked, and unstable data was rejected, as described in reference to FIGS. 19A-19B. Subject-specific baseline temperature values were also determined as described in FIGS. 20A-20B.

For example, FIG. 22 shows temperature and capacitance data collected from a subject for eight days for a subject wearing a thermal sensing device that includes a sensor unit configured to include both capacitance and temperature sensing. In FIG. 22, the raw data shows a larger variation in the temperature data (which typically varied between 22 degrees C. (when outside of the mouth) and 37 degrees (when worn). The horizontal dashed lines show the expected temperature range when the device is worn in the mouth. Thus, FIG. 22 shows periods in which the temperature was relatively stable (between 36-38 degrees) and periods where the signal indicated that the temperature was lower, e.g., likely due to removal of the device from the mouth. The capacitance data agrees with this conclusion, showing a change in capacitance corresponding to the low-temperature regions, likely due to removal from the body.

These method and apparatuses may also generally allow comparison between the actual temperature of the sensor unit (e.g., the thermal sensing device) and the baseline as described above. FIG. 23 is graph showing a comparison of baseline temperature and body temperature for a subject wearing an intraoral appliance. In FIG. 23, the baseline remained relatively stable at around 36 degrees C., while the body as measured from the sensor unit, during periods when, as described in FIGS. 19A-19B, the sensor unit was confirmed to be in the subject's mouth.

FIG. 24 is graph that also shows a comparison between the baseline and sensed temperature using the thermal sensing device. In FIG. 24, the graph shows the duration (in hours) each day that the temperature as measured by the thermal sensing device was either below (“low temperature duration”) at (“normal temperature duration”) or above (“high temperature duration”) the baseline temperature. Thus, the graph illustrates the duration of periods of low, normal and high temperature, as compared to baseline body temperature, using an intraoral appliance including a sensor unit that is worn by the subject.

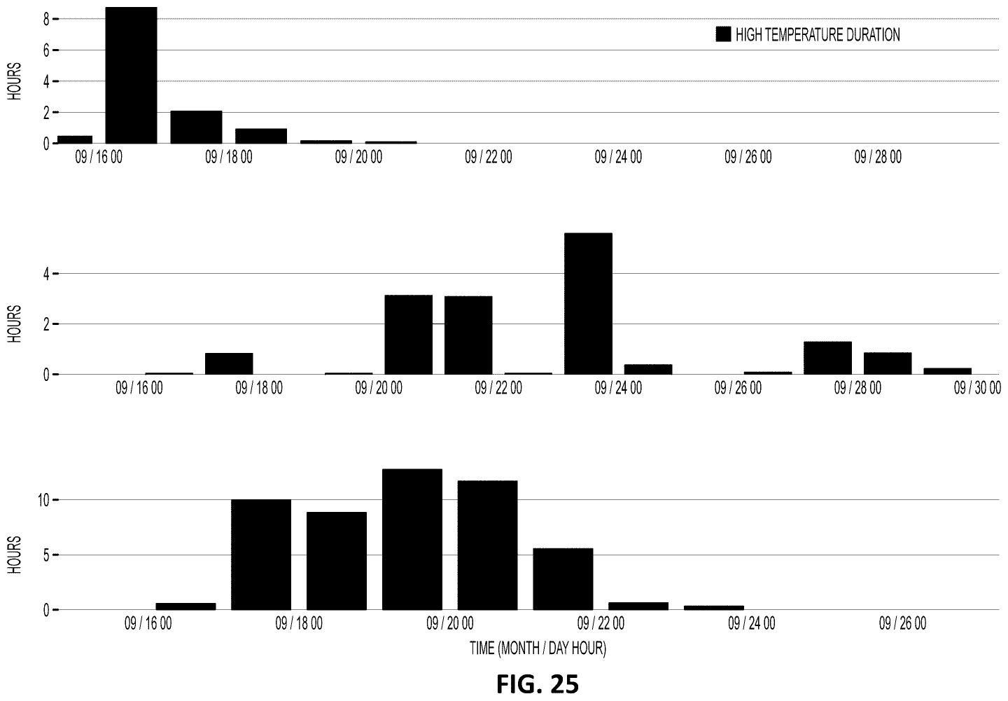

These methods and apparatuses were examined for use with subjects having an elevated temperature, and was shown (e.g., in FIG. 25) to track the periods of high temperature showing an elevation in body temperature above baseline indicating a fever or other stress. In FIG. 25 the bars represent the number of hours that this particular subject had an oral temperature as measured by the thermal sensing device that was greater than the baseline temperature (“high temperature duration”). The graph shows a period of three weeks.

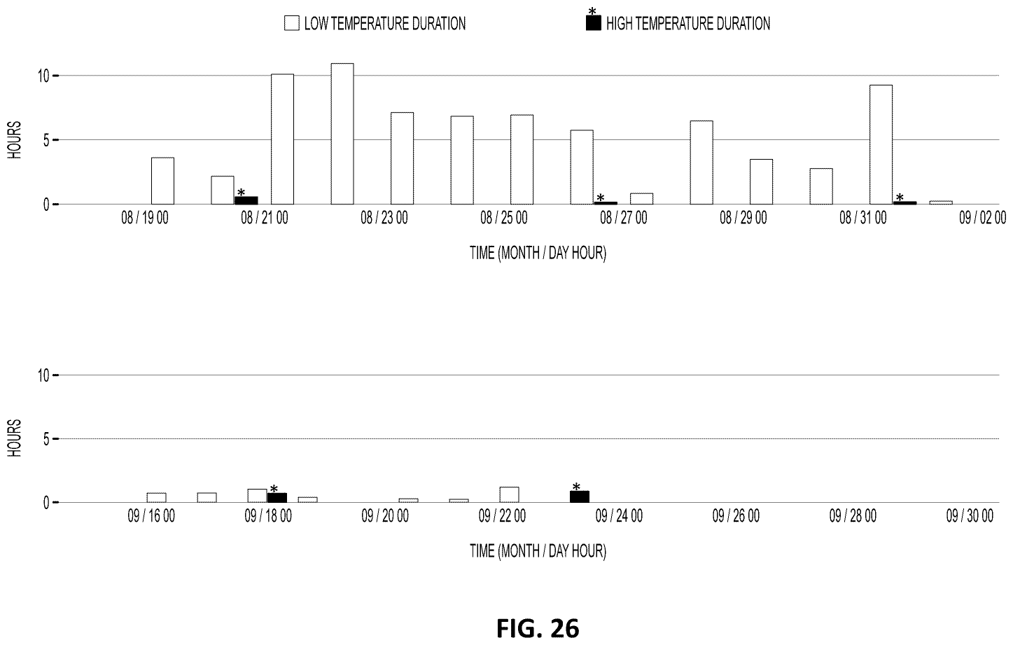

Interestingly, the thermal sensing devices described herein were able to detect differences in healthy vs. less healthy subjects based on the observed body temperature. In particular, in a subset of subjects that were correlated with regular exercise, the time in which these fit subjects experienced an oral body temperature that was less than the baseline body temperature as determined above (e.g., FIGS. 20A-20B) was greater than in subjects that did not regularly exercise or did not exercise as much. In FIG. 26, the first week of data collected showed greater duration of periods of low temperature, indicating that physical activity may result in detectable lower body temperature. Thus the use of the baseline as a comparison reference, and in particular a subject-specific baseline, may allow for determination of one or more indicators of health and fitness, as described above. In some embodiments, a fitness score may be calculated and provided to the user.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for case of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1-116. (canceled)

117. A system for intraoral sensing, the system comprising:
a sensor unit comprising a housing having a flange extending at least partially around a base of the housing, wherein the housing at least partially encloses a sensor;
a processor that is configured to receive physiological readings from the sensor; and
an intraoral device comprising a body forming a tooth-receiving cavity that is configured to fit over a subject's teeth, and an opening through a wall of the tooth receiving cavity, wherein the opening is configured to receive the housing of the sensor unit, so that the flange engages with an inner surface of the tooth-receiving cavity to secure the sensor unit in position when the intraoral device is worn on the teeth.

118. The system of claim 117, wherein the opening is configured to hold the base of the housing flush with the inner surface of the intraoral device.

119. The system of claim 117, wherein the opening is configured to hold the base of the housing in parallel with the inner surface of the intraoral device when the intraoral device is worn.

120. The system of claim 117, wherein the opening extends through a buccal side of the intraoral device.

121. The system of claim 117, wherein the opening extends through a lingual side of the intraoral device.

122. The system of claim 117, wherein the opening further comprises a recessed region that is recessed into the inner surface of the tooth-receiving cavity and configured to receive the flange of the intraoral device.

123. The system of claim 117, wherein the opening is configured to seal to the housing of the sensor unit.

124. The system of claim 117, wherein the sensor unit is configured to measure a temperature having a sensitivity of 0.1 degree Celsius or greater.

125. The system of claim 117, further wherein the sensor unit further comprises one or more accelerometers configured to detect movement data and to store, transmit or store and transmit the movement data.

126. The system of claim 117, wherein sensor unit is configured to continuously record temperature readings.

127. The system of claim 117, wherein the intraoral device comprises one or more of: an aligner, a retainer, and/or a palatal expander.

128. The system of claim 117, wherein the processor is configured to store, transmit or store and transmit thermal data.

129. The system of claim 128, wherein the processor is configured to determine an illness condition based on the thermal data.

130. The system of claim 128, wherein the processor is configured to determine a fitness metric based on the thermal data.

131. The system of claim 128, wherein the processor is configured to determine an ovulation or pregnancy condition based on the thermal data.

132. The system of claim 128, wherein the processor is configured to determine compliance with a dietary plan and/or fitness plan based on the thermal data.

133. The system of claim 128 wherein the processor is configured to determine compliance based on the thermal data.

134. The system of claim 128, wherein the processor is configured to identify one or more sleep/wake cycles based on the thermal data and determine a sleep abnormality condition based on the identified sleep wake cycles.

135. The system of claim 128, wherein the processor is configured to identify an infection condition based on the thermal data.

136. A method, the method comprising:
inserting a sensor unit comprising a housing at least partially enclosing a sensor and having a flange extending at least partially around a base of the housing through an opening on an intraoral mount;
securing the intraoral mount within a subject's oral cavity, wherein the intraoral mount is configured to fit over a subject's teeth, and an opening through a wall of the tooth receiving cavity, wherein the opening is configured to receive the housing of the sensor unit, so that the flange engages with an inner surface of the tooth-receiving cavity to secure the sensor unit in position when the intraoral device is worn on the teeth; and
receiving one or more physiological readings from the sensor.

137. The method of claim 136, wherein the intraoral mount comprises one of: a patient-removable aligner, an upper arch expander, a lower arch expander and/or a retainer.

138. The method of claim 136, wherein receiving one or more physiologic readings comprises receiving temperature from the sensor.

139. A method, the method comprising:
receiving temperature sensor data from a sensor unit worn in a subject's oral cavity on an intraoral mount;
confirming that the subject is wearing the sensor unit;
confirming that the temperature reading does not correspond to an environmental influence;
determining if a subject has an elevated temperature by comparing the temperature sensor data to an elevated temperature detection threshold based; and
triggering an alert indicating that the subject has an elevated temperature if the temperature sensor data is greater than the elevated temperature detection threshold.
